(19) United States
(12) Patent Application Publication
(10) Pub. No.: US 2014/0308258 A1
(43) Pub. Date: Oct. 16, 2014

(54) ENHANCED MICROBIAL STRAINS FOR THE PREVENTION AND TREATMENT OF METHICILLIN-RESISTANT STAPHYLOCOCCUS AUREUS AND METHICILLIN-RESISTANT STAPHYLOCOCCUS EPIDERMIS INFECTION

(71) Applicant: Charles Joseph Matthews, Raleigh, NC (US)

(72) Inventor: Charles Joseph Matthews, Raleigh, NC (US)

(21) Appl. No.: 14/251,187

(22) Filed: Apr. 11, 2014

Related U.S. Application Data
(60) Provisional application No. 61/811,345, filed on Apr. 12, 2013.

(51) Int. Cl. A61K 36/064; A61K 35/74

(52) U.S. Cl. CPC A61K 36/064; A61K 35/747
USPC 424/93.21; 435/254.2; 435/254.21; 435/252.3; 424/93.2

(57) ABSTRACT

The present invention provides enhanced microorganisms useful for inhibiting and/or preventing the proliferation of pathogenic microorganisms such as methicillin-resistant Staphylococcus aureus and methicillin-resistant Staphylococcus epidermidis.

### RELATED APPLICATION

This application claims the benefit of priority under 35 U.S.C. §119(e) to U.S. Provisional Patent Application No. 61/811,345, filed Apr. 12, 2013, the disclosure of which is incorporated herein by reference in its entirety.

### FIELD OF THE INVENTION

The present invention relates to enhanced microorganisms useful for inhibiting and/or preventing the proliferation of pathogenic microorganisms such as methicillin-resistant Staphylococcus aureus and methicillin-resistant Staphylococcus epidermidis.

### BACKGROUND

Infections caused by drug-resistant microorganisms often fail to respond to traditional therapies, which leads to prolonged illness and increased risk of death.

There is thus a need for alternative therapies to combat drug-resistant microorganisms.

### SUMMARY OF THE INVENTION

A first aspect of the present invention is a method of producing an enhanced microbial strain, comprising culturing a first microbial strain in the presence of a second microbial strain under conditions sufficient to produce an enhanced microbial strain derived from the first microbial strain and selecting said enhanced microbial strain.

A second aspect of the present invention is an enhanced microbial strain.

A third aspect of the present invention is a medicament comprising an enhanced microbial strain.

A fourth aspect of the present invention is a wound dressing comprising an enhanced microbial strain.

A fifth aspect of the present invention is a disinfectant comprising an enhanced microbial strain.

A sixth aspect of the present invention is a method of inhibiting and/or preventing colonization of a substrate by one or more pathogenic microbial strains, comprising applying an enhanced microbial strain to said substrate.

A seventh aspect of the present invention is a method of inhibiting and/or preventing proliferation of one or more pathogenic microbial strains on a substrate, comprising applying an enhanced microbial strain to said substrate.

An eighth aspect of the present invention is a method of killing one or more pathogenic microbial strains on a substrate, comprising applying an enhanced microbial strain to said substrate.

A ninth aspect of the present invention is a method of enhancing restoration of the normal microbial environment of a substrate, comprising applying an enhanced microbial strain to said substrate.

A tenth aspect of the present invention is a method of treating a disorder caused by one or more pathogenic microbial strains to a subject in need thereof, comprising administering to said subject a therapeutically effective amount of an enhanced microbial strain.

An eleventh aspect of the invention is a method of enhancing the likelihood of survival of a wounded subject, comprising administering to said subject a therapeutically effective amount of an enhanced microbial strain.

A twelfth aspect of the invention is a method of inhibiting and/or preventing the onset of sepsis in a subject in need thereof, comprising administering to said subject a prevention effective amount of an enhanced microbial strain.

A thirteenth aspect of the invention is a method of enhancing one or more aspects of wound healing in a subject in need thereof, comprising administering to said subject a therapeutically effective amount of an enhanced microbial strain.

### DETAILED DESCRIPTION

The present invention is explained in greater detail below. This description is not intended to be a detailed catalog of all the different ways in which the invention may be implemented or of all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein, which do not depart from the instant invention, will be apparent to those skilled in the art in light of the instant disclosure. Hence, the following specification is intended to illustrate some particular embodiments of the invention, and not to exhaustively specify all permutations, combinations and variations thereof.

The terminology used herein is for the purpose of describing particular embodiments only and is not intended to be limiting of the invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly format sense unless expressly so defined herein. Well-known functions or constructions may not be described in detail for brevity and/or clarity.

As used herein, the singular forms “a”, “an” and “the” are intended to include t plural forms as well, unless the context clearly indicates otherwise.

As used herein, the term “about,” when used in reference to a measurable value such as an amount of mass, dose, time, temperature, and the like, is meant to encompass variations of 20%, 10%, 5%, 1%, 0.5%, or even 0.1% of the specified amount.

As used herein, the term “adjuvant treatment” refers to a treatment that modifies the effect(s) of one or more other treatments (e.g., one or more pharmaceutical agents). For example, the application of one or more enhanced microbial strains of the present invention may enhance the effectiveness of a pharmaceutical agent. In some embodiments, the application of one or more enhanced microbial strains of the present invention may reduce or eliminate the need for one or more other treatments (e.g., one or more pharmaceutical agents).

As used herein, the term “and/or” includes any and all combinations of one or more of the associated listed items.

As used herein, the term “commensal” refers to a microbe whose presence neither harms nor benefits a host organism (e.g., a subject).

As used herein, the terms “comprise,” “comprises,” “comprising, ” “include,” “includes” and “including” specify the presence of stated features, integers, steps, operations, elements, and/or components, but do not preclude the presence or addition of one or more other features, integers, steps, operations, elements, components, and/or groups thereof.

As used herein, the term “consists essentially of” (and grammatical variants thereof), as applied to the medicaments and methods of the present invention, means that the medicaments/methods may contain additional components so long as the additional components do not materially alter the medicament/method. The term “materially alter,” as applied to a medicament/method, refers to an increase or decrease in the effectiveness of the medicament/method of at least about 20% or more. For example, a component added to a medicament of the present invention would “materially alter” the medicament if it increases or decreases the medicament's ability to inhibit and/or prevent the growth of a pathogenic microbial strain by at least about 50%.

As used herein, the term “effective amount” refers to an amount that imparts a desired effect. In some embodiments, the desired effect comprises a therapeutic effect and/or a prophylactic effect.

As used herein, the term “emulsion” refers to a suspension or dispersion of one liquid within a second immiscible liquid. In some embodiments, the emulsion is an oil-in-water emulsion or a water-in-oil emulsion. In some embodiments, “emulsion” may refer to a material that is a solid or semi-solid at room temperature and is a liquid at body temperature (about 37° C.).

As used herein, the term “enhanced microbial strain” refers to a non-naturally occurring microbial strain that possesses at least one desired trait. In some embodiments, the enhanced microbial strain possesses a desired trait that was absent in the strain from which it was derived. In some embodiments, both the enhanced microbial strain and the microbial strain from it was derived possess the desired trait, but the enhanced microbial strain has a stronger phenotype for the desired trait. Any suitable microbe may be enhanced, including, but not limited to, prokaryotic microorganism and eukaryotic microorganisms. In some embodiments, the enhanced microbial strain is a bacterial strain, a yeast strain or a mold strain.

As used herein, the term “excipient” refers to any substance that is used as a carrier, diluent, binder, or vehicle for delivery of a therapeutic agent to a subject and/or added to a medicament to improve its handling or storage properties or to permit or facilitate formation of a compound or composition into a unit dosage form for administration. Excipients may provide a variety of functions and may be described as wetting agents, buffering agents, suspending agents, lubricating agents, emulsifiers, disintegrants, absorbents, preservatives, surfactants, colorants, flavorants, and sweeteners. Pharmaceutically acceptable excipients are well known in the pharmaceutical arts and are described, for example, in Ansel's PHARMACEUTICAL DOSAGE FORMS AND DRUG DELIVERY SYSTEMS (9th Ed., Lippincott Williams and Wilkins (2010)), HANDBOOK OF PHARMACEUTICAL EXCIPIENTS (3rd Ed., American Pharmaceutical Association, Washington, D.C. (2000)), Remington's PHARMACEUTICAL SCIENCES (20th Ed., Mack Publishing Co., Easton, Pa. (2000)) and REMINGTON: THE SCIENCE AND PRACTICE OF PHARMACY (21st Ed., Lippincott Williams & Wilkins (2005)).

As used herein, the terms “inhibit,” “inhibiting,” and “inhibition” (and grammatical variants thereof) refer to delaying the onset of and/or reducing the severity of a disease, disorder and/or a clinical symptom(s) in a subject relative to what would occur in the absence of the methods of the present invention and/or to delaying the onset of and/or reducing the magnitude of an event in the life cycle of a microbial strain (e.g., colonization and/or proliferation) relative to what would occur in the absence of the methods of the present invention. In some embodiments, inhibition is complete, resulting in the total absence of the disease, disorder and/or clinical symptom(s) (e.g., a total absence of growth of a pathogenic microbial strain). In some embodiments, inhibition is partial, resulting in reduced severity and/or delayed onset of the disease, disorder and/or clinical symptom(s) (e.g., a reduced proliferation rate of a pathogenic microbial strain).

As used herein, the terms “inhibitory substance” refers to any substance that inhibits and/or prevents the colonization of a substrate (e.g., human tissue) by one or more microbial strains, that inhibits and/or prevents the proliferation of one or more microbial strains and/or that kills one or more microbial strains. In some embodiments, the inhibitory substance inhibits and/or prevents the colonization of a substrate (e.g., human tissue) by one or more pathogenic microbial strains, inhibits and/or prevents the proliferation of one or more pathogenic microbial strains and/or kills one or more pathogenic microbial strains.

As used herein, the term “non-naturally occurring microbial strain” refers to a microbial strain that does not naturally exist in nature. A non-naturally occurring microbial strain may be produced by any method known in the art, including, but not limited to, transforming a naturally occurring microbial strain, crossing a naturally occurring microbial strain with a non-naturally occurring microbial strain and culturing a naturally occurring microbial strain under non-naturally occurring conditions. In some embodiments, the non-naturally occurring microbial strain comprises one of more heterologous nucleotide sequences. In some embodiments, the non-naturally occurring microbial strain comprises one or more non-naturally occurring copies of a naturally occurring nucleotide sequence (i.e., extraneous copies of a gene that naturally occurs in the microbial strain from which the non-naturally occurring microbial strain was derived) and/or lacks a naturally occurring nucleotide sequence. In some embodiments, the non-naturally occurring microbial strain comprises a non-natural combination of two or more naturally occurring nucleotide sequences (i.e., two or more naturally occurring genes that do not naturally occur in the same microbial strain).

As used herein, the term “normal microbial environment” refers to the microbial flora that typically inhabit a given substrate. In some embodiments, the normal microbial environment comprises the microbial strains that typically inhabit a particular human tissue in the absence of treatment with antibioticsantivirals and/or in the absence of a wound to said tissue. In some embodiments, the normal microbial environment comprises the microbial strains that typically inhabit the tissue of a given subject. In some embodiments, the normal microbial environment comprises the microbial strains that typically inhabit the tissue of one or more control subjects.

As used herein, the term “pathogenic microbial strain” refers to a microbial strain that has the potential to cause or produce disease. Any suitable microbe may be pathogenic, including, but not limited to, prokaryotic microorganisms and eukaryotic microorganisms. In some embodiments, the pathogenic microbial strain is a bacterial strain, a yeast strain or a mold strain

As used herein, “pharmaceutically acceptable” means that the material is suitable for administration to a subject and will allow desired treatment to be carried out without giving rise to unduly deleterious side effects. The severity of the disease and the necessity of the treatment are generally taken into account when determining whether any particular side effect is unduly deleterious.

As used herein, the terms “prevent,” “preventing,” and “prevention” (and grammatical variants thereof) refer to avoiding the onset of a disease, disorder and/or a clinical symptom(s) in a subject relative to what would occur in the absence of the methods of the present invention and/or to avoiding an event in the life cycle of a microbial strain (e.g., colonization and/or proliferation) relative to what would occur in the absence of the methods of the present invention. In some embodiments, prevention is complete, resulting in the total absence of the disease, disorder and/or clinical symptom(s) (e.g., a total absence of growth of a pathogenic microbial strain). In some embodiments, prevention is partial, resulting in avoidance of some aspects of the disease, disorder and/or clinical symptom(s) (e.g., colonization by a pathogenic microbial strain but no subsequent proliferation).

As used herein, the term “prevention effective amount” (and grammatical variants thereof) refers an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some benefit is provided to the subject.

As used herein, the term “probiotic” refers to microbe whose presence confers one or more health benefits to a host organism (e.g., a subject). In some embodiments, the present of the probiotic microbe benefits the host by inhibiting the proliferation of one or more pathogenic microbes (e.g., by competing with the pathogenic microbe(s) for resources and/or by producing a substance that inhibits the proliferation of the pathogenic microbe(s)).

As used herein, the term “subject” refers to both human subjects and animal subjects, including, but not limited to, mice, rats, rabbits, cats, dogs, pigs, horses, monkeys, apes, etc.

The subject may be male or female. That subject may be of any suitable age, including infant, juvenile, adolescent, adult and geriatric ages. In some embodiments, the methods, devices and systems of the present invention may be used to induce physiological and/or psychological responses in a subject for medically diagnostic and/or therapeutic purposes. For example, the methods, devices and systems of the present invention may be used to diagnose and/or treat mammalian subjects, such as mice, rats, pigs and monkeys, for medical research or veterinary purposes.

As used herein, the terms “therapeutically effective amount” and “therapeutically acceptable amount” (and grammatical variants thereof) refer to an amount that will elicit a therapeutically useful response in a subject. The therapeutically useful response may provide some alleviation, mitigation, or decrease in at least one clinical symptom in the subject. The terms also include an amount that will prevent or delay at least one clinical symptom in the subject and/or reduce and/or delay the severity of the onset of a clinical symptom in a subject relative to what would occur in the absence of the methods of the invention. Those skilled in the art will appreciate that the therapeutically useful response need not be complete or curative or prevent permanently, as long as some benefit is provided to the subject.

As used herein, the terms “treatment,” “treat,” and “treating” refer to reversing, alleviating, reducing the severity of delaying the onset of or inhibiting the progress of a disease or disorder as described herein. In some embodiments, treatment comprises reversing, alleviating, reducing the severity of, delaying the onset of, inhibiting the progress of and/or preventing at least one symptom of a disease or disorder as described herein (e.g., inhibiting and/or preventing proliferation of one or more pathogenic microbial strains in a wound). In some embodiments, treatment may be administered after one or more symptoms have developed (e.g., following wound infection by one or more pathogenic microbial strains). In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved—for example, to prevent or delay their recurrence. Treatment may be as an adjuvant treatment as further described herein.

As used herein, the term “treatment effective amount” (and grammatical variants thereof) refers to an amount that is sufficient to provide some improvement or benefit to the subject. Alternatively stated, a “treatment effective amount” is an amount that will provide some alleviation, mitigation, decrease, or stabilization in at least one clinical symptom in the subject. Those skilled in the art will appreciate that the therapeutic effects need not be complete or curative, as long as some benefit is provided to the subject.

While certain aspects of the present invention will hereinafter be described with reference to embodiments thereof, it will be understood by those of ordinary skill in the art that various changes in form and details may be made therein without departing from the spirit and scope of the present invention as defined by the claims.

All publications, patent applications, patents, and other references mentioned herein are incorporated by reference in their entirety.

The present invention provides enhanced microbial strains and methods of producing and using the same.

Enhanced microbial strains of the present invention may be derived from any suitable microbial strain, including, but not limited to, bacterial strains, mold strains and yeast strains.

Enhanced microbial strains of the present invention may be derived from any suitable bacterial strain, including, but not limited to, probiotic bacterial strains and commensal bacterial strains. In some embodiments, the enhanced microbial strain is derived from a bacterial strain that produces lactic acid. In some embodiments, the enhanced microbial strain is derived from a bacterial strain belonging to a genus selected from the group consisting of Carnobacterium (e.g., C. alterfunditum, C. divergens C. funditum, C. gallinarium, C. inhibens, C. jeotgali, C. maltaramatieum, C. mobile, C. piscicola, C. pleistocenium and/or C. viridans), Enterococcus (e.g., E. aquimarinus, E. asini, E. avium, E. caccae, E. camelliae, E. caniniestini, E. canis, E. casseloflavus, E. cecorum, E. columbae, E. devriesei, E. dispar, E. durans, E. faecalis, E. faecium, E. flavescens, E. gallnarum, E. gilvus, E. haemoperoxidus, E. hermanniensis, E. hirae, E. iatlicus, E. lactis, E. maodoratus, E. moraviensis, E. mundatii, E. pallens, E. phoeniculicola, E. plantarum, E. porcinus, E. pseudoavium, E. quebecensis. E. rallitiosus, E. rani, E. rivorum, E. saccharolyticus, E. saccharomimimus, E. seriolicida, E. silesiacus, E. solitarius, E. sulfureus, E. termitis, E. thailandicus, E. ureasiticus, E. vikkiensis and/or E. villorum), Lactobacillus (e.g., L. acetotolerans, L. acidifarinae, L acidipiscis, L. acidophilus, L, agilis, L. algidus, L. alimentarius, L. amylolyticus, L. amylophilus, L. amylotrophicus, L. amylovorus, L. animalis, L. antri, L. apodemi, L. aquaticus, L. arizonensis, L. aviarius, L. bavaricus, L. bifermentans, L. bobalius, L. brantae, L. brevis, L. buchneri, L. bulgarius, L. caaonum, L. camelliae, L. capillatus, L. carnis, L. casei, L. catenaformis, L. cellobiosus, L. ceti, L. coleohominis, L. collinoides, L. composti, L. concavus, L. confusus, L. coryniformis, L. crispatus, L. crustorum, L. curvatus, L. cypricasei, L. delbrueckii, L. dextrinicus, L. diolovorans, L. divergens, L. durianis, L. equi, L. equicursoris, L. equigenerosi, L. fabifermentans, L. farciminis, L. farraginis, L. ferintoshensis, L. fermentum, L. floricola, L. florum, L. fronicalis, L. fructivorans, L. fructosus, L. frumenti, L. fuchuensis, L. futsaii, L. gallinarium, L. gasseri, L. gastricus, L. ghanensis, L. gigeriorum, L. graminis, L. halotolerans, L. hammesii, L. hamsteri, L. harbinensis, L. hayakitensis, L. helveticus, L. heterochii, L. hilgardii, L. hominis, L. homohiochii, L. hordei, L. iners, L. ingluviei, L. intestinalis, L. jensenii, L. johnsonii, L. katixensis, L. kandleri, L. kerfiranofaciens, L. kefirgranum, L. kefiri, L. kimchicus, L. kimchii, L. kisonensis, L. kitasatonis, L. koreensis, L. kunkeei, L. lactis, L. leichmannii, L. lindneri, L. malefermentans, L. mali, L. maltaromicus, L. manihotivorans, L. mindensis, L. minor, L. minutus, L. mucosae, L. murinus, L. nagelii, L. namurensis, L. nantensis, L. nasuensis, L. nodensis, L. odoratitofui, L. oeni, L. oligolermentans, L. oris, L. otakiensis, L ozensis, L. panis, L. pantheris, L. parabrevis, L. parabuchneri, L. paracasei, L. paracollinoides, L. parafarraginis, L. parakefiri, L. paralimentarius, L. paraplantarum, L. pasteurii, L. paucivorans, L. pentosus, L. perolens, L. piscicola, L. plantarum, L. pobuzihii, L. pontis, L. psittaci, L. rapi, L. rennini, L. reuteri, L. rhamnosus, L. rimae, L. rogosae, L. rossiae, L. ruminis, L. saerimneri, L. sakei, L. salivarius, L. sanfranciscenis, L. saniviri, L. satsumensis, L. secaliphilus, L. selangorensis, L. senioris, L. senmaizukei, L. sharpeae, L. siliginis, L. similis, L. sobrius, L. spicheri, L. sporogenes, L. sucicola, L. suebicus, L. sunkii, L. suntoryeus, L. taiwanensis, L. thailandensis, L. thermotolerans, L. trchodes, L. tucceti, L. uli, L. ultunensis, L. uvarium, L. vaccinostercus, L. vaginalis, L. versmoldensis, L. vini, L. viridescens, L. vitulinus, L. xiangfangensis, L. xylosus, L. yamanashiensis, L. zeae and/or L. zymae), Lactococcus (e.g., L. chugangensis, L. fujiensis, L. garvieae, L. lactis, L. piscium, L. plantarum and/or L. raffinolactis), Leuconostoc (e.g., L. amelibiosum, L. argentinum, L. carnosum, L. citreum, L. cremoris, L. dextranicum, L. durionis, L. fallax, L. ficulneum, L. fructosum, L. gasicomitatum, L. geidum, L. holzapfelii, L. inhae, L. kimchii, L. lactis, L. mesenteroides, L. miyukkimchii, L. oeni, L. palmae, L. paramesenteroides, L. pseudaficulnemn and/or L. pseudomesenteroides), Oenococcus (e.g., O. kitaharae and/or O. oeni), Pediococcus (e.g., P. argentinicus, P. cellicola, P. claussenii, P. damnosus, P. dextrinicus, P. ethanolidruans, P. halophilus, P. inopinatus, P. lolii, P. parvulus, P. pentosaceus, P. siamensis, P. stilesii and/or P. urinaeequi), Streptococcus (e.g., S. acidominimus, S. adjacens, agalactiae, S. alactolyticus, S. anginosus, S. australius, S. bovis, S. caballi, S. canis, S. caprinus, S. castoreus, S. cecorum, S. constellatus, S. cremoris, S. criceti, S. cristatus, S. defectivus, S. dentapri, S. dentirousetti, S. devriesei, S. didelphis, S. difficilis, S. downei, S. durans, S. dysgalactiae, S. entericus, S. equi, S. equinus, S. faecalis, S. faecium, S. ferus, S. gallinaceus, S. gallnarium, S. gallolyticus, S. garvieae, S. gordonii, S. halichoeri, S. hansenii, S. henryi, S. hyointestinalis, S. hyovaginalis, S. ictaluri, S. infantarius, S. infantis, S. iniae, S. intermedius, S. intestinals, S. lactorius, S. lactis, S. luteitensis, S. macacae, S. macedonicus, S. marimammalium, S. massiliensis, S. merionis, S. minor, S. mitis, S. morbillorum, S. mutans, S. oligofermentans, S. oralis, S. orisratti, S. orisuis, S. ovis, S. parasanguinis, S. parauberis, S. paravuluvs, S. pasteurianus, S. peroris, S. phocae, S. plantarum, S. plemorphus, S. pluranimalium, S. plurextorum, S. pneumoniae, S. porci, S. porcinus, S. porcorum, S. pseudopneumoniae, S. pseudoporcinus, S. pyogenes, S. raffinolactis, S. ratti, S. rupicaprae, S. saccharolyticus, S. salivarius, S. sanguinis, S. shiloi, S. sinensis, S. sobrinus, S. suis, S. thermophilus, S. thoraltensis, S. tigurinus, S. uberis, S. urinalis, S. ursoris, S. vestibularis and/or S. waius), Tetragenococcus (e.g., T. halophilus, T. koreensis, T. muriaticus, T. osmophilus and/or T. solitarius), Vagococcus (e.g., V. acidifermentans, V. carniphilus, V. elongatus, V. fessus, V. fluvialis, V. lutrae, V. penaei and/or V. salmoninarum) and Weissella (e.g., W. beninensis, W. ceti, W. cibaria, W. confusa, W. fabaria, W. ghanensis, W. halotolerans, W. hellenica, W. kandleri, W. kimchii, W. koreensis, W. minor, W. paramesenteroides, W. soli, W. thailandensis and/or W. viridescens). In some embodiments, the enhanced microbial strain is derived from a bacterial strain belonging to a species selected from the group consisting of Lactobacillus acidophilus, Lactobacillus brevis, Lactobacillus buchneri, Lactobacillus casei, Lactobacillus curvatus, Lactobacillus delbrueckii, Lactobacillus fermentum, Lactobacillus helveticus, Lactobacillus plantarum, Lactobacillus reuteri, Lactobacillus sakei, Lactobacillus sporogenes and Lactobacillus salivarius. In some embodiments, the enhanced microbial strain is not derived from Bacillus coagulans.

Enhanced microbial strains of the present invention may be derived from any suitable mold strain, including, but riot limited to, probiotic mold strains and commensal mold strains.

Enhanced microbial strains of the present invention may be derived from any suitable yeast strain, including, but not limited to, probiotic yeast strains and commensal yeast strains. In some embodiments, the enhanced microbial strain is derived from a yeast strain belonging to a genus selected from the group consisting of Brettanomyces (e.g., B. anomalus, B. bruxellensis, B. custersianus, B. naardenensis and/or B. nanus), Candida (e.g., C. stellata), Dekkera (e.g., D. anomala and/or D. bruxellensis), Saccharomyces (e.g., S. cerevisiae, S. bayanus, S. boulardii and/or S. pastorianus), Schizosaccharomyces (e.g., S. pombe), Torulaspora (e.g., T. delbrueckii), Torulopsis and Zygosaccharomyces (e.g., Z. bailii). In some embodiments, the enhanced microbial strain is derived from a yeast strain belonging to a species selected from the group consisting of Brettanomyces anomalus, Brettanomyces bruxellensis, Brettanomyces custersianus, Brettanomyces naardenensis, Brettanomycesnanus, Dekkera anomala, Dekkera bruxellensis, Saccharomyces cerevisiae, Saccharomyces bayanus, Saccharomyces boulardii and Saccharomyces pastorianus.

Enhanced microbial strains of the present invention may possess any suitable traits, In some embodiments, the enhanced microbial strain possesses one or more desired traits, such as the ability to inhibit and/or prevent the growth and/or proliferation of one or more pathogenic microbial strains. In sonic embodiments, the enhanced microbial strain possesses at least one desired trait that is absent in the microbial strain from which it was derived. For example, in some embodiments, the enhanced microbial strain demonstrates an increased ability to inhibit and/or prevent the colonization of a substrate (e.g., human tissue) by one or more pathogenic microbial strains, an increased ability to inhibit and/or prevent the proliferation of one or more pathogenic microbial strains and/or an increased ability to kill one or more pathogenic microbial strains, as compared to the microbial strain from which it was derived. Thus, in some embodiments, the enhanced microbial strain is more adept at competing with one or more pathogenic microbial strains than is the microbial strain from which it was derived. In some embodiments, the enhanced microbial strain's increased ability to compete with the pathogenic microbial strain(s) is due, at least in part, to an increased ability, as compared to the microbial strain from which it was derived, to produce one or more inhibitory substances. In some embodiments, the enhanced microbial strain's increased ability to compete with the pathogenic microbial strain(s) is due, at least in part, to an increased ability, as compared to the microbial strain from which it was derived, to raise the pH of its environment (e.g., by releasing minerals such calcium and magnesium into its environment). In some embodiments, the enhanced microbial strain's increased ability to compete with the pathogenic microbial strain(s) is due, at least in part, to an increased ability, as compared to the microbial strain from which it was derived, to lower the pH of its environment (e.g., by releasing carbon dioxide into its environment). In some embodiments, the enhanced microbial strain's increased ability to compete with the pathogenic microbial strain(s) is due, at least in part, to an increased ability, as compared to the microbial strain from which it was derived, to raise the alkalinity of its environment (e.g., by releasing minerals such calcium and magnesium into its environment). In some embodiments, the enhanced microbial strain's increased ability to compete with the pathogenic microbial strain(s) is due, at least in part, to an increased ability, as compared to the microbial strain from which it was derived, to lower the alkalinity of its environment (e.g., by releasing carbon dioxide into its environment). In some embodiments, the enhanced microbial strain's increased ability to compete with the pathogenic microbial strain(s) is due, at least in part, to an increased ability, as compared to the microbial strain from which it was derived, to raise the level of one or more gases in its environment (e.g., carbon dioxide, methane and/or oxygen). In some embodiments, the enhanced microbial strain's increased ability to compete with the pathogenic microbial strain(s) is due, at least in part, to an increased ability, as compared to the microbial strain from which it was derived, to lower the level of one or more gases in its environment (e.g., carbon dioxide, methane and/or oxygen). In some embodiments, the enhanced microbial strain's increased ability to compete with the pathogenic microbial strain(s) is due, at least in part, to an increased ability, as compared to the microbial strain from which it was derived, to promote the colonization of a substrate (e.g., human tissue) by one or more other microbial strains (e.g., one or more probiotic microbial strains). In some embodiments, the enhanced microbial strain's increased ability to compete with the pathogenic microbial strain(s) is due, at least in part, to an increased ability, as compared to the microbial strain from which it was derived, to promote the growth and/or proliferation of one or more other microbial strains (e.g., one or more probiotic microbial strains). In some embodiments, the enhanced microbial strain's increased ability to compete with the pathogenic microbial strain(s) is due, at least in part, to an increased ability, as compared to the microbial strain from which it was derived, to inhibit the death of one or more other microbial strains (e.g., one or more probiotic microbial strains). In some embodiments, the enhanced microbial strain's increased ability to compete with the pathogenic microbial strain(s) is due, at least in part, to an increased ability, as compared to the microbial strain from which it was derived, to uptake and/or utilize one or more resources (e.g., one or more resources required for the growth and/or proliferation of the pathogenic microbial strain(s)). In some embodiments, the enhanced microbial strain's increased ability to compete with the pathogenic microbial strain(s) is due, at least in part, to an increased ability, as compared to the microbial strain from which it was derived, to survive and/or proliferate in a desiccating environment. In some embodiments, the enhanced microbial strain's increased ability to compete with the pathogenic microbial strain(s) is due, at least in part, to an increased ability, as compared to the microbial strain from which it was derived, to survive and/or proliferate in a nutrient-deficient environment.

Enhanced microbial strains of the present invention may inhibit and/or prevent the colonization of a substrate (e.g., human tissue) by any suitable pathogenic microbial strain, including, but not limited to, bacterial strains, mold strains and yeast strains.

Enhanced microbial strains of the present invention may inhibit and/or prevent the colonization of a substrate (e.g., human tissue) by any suitable bacterial strain, including, but not limited to, pathogenic bacterial strains. In some embodiments, the enhanced microbial strain inhibits and/or prevents the colonization of a substrate (e.g., human tissue) by one or more bacterial strains belonging to a genus selected from the group consisting of Acinetobacter (e.g., A. baumannii), Clostridium (e.g., C. difficile), Enterococcus (e.g., E. faecalis and/or E. faecium), Escherichia (e.g., E. coli), Mycobacterium (e.g., M. tuberculosis), Pseudomonas (e.g., P. aeruginosa), Salmonella, Staphylococcus (e.g., S. aureus and/or S. epiderimis) and Streptococcus (e.g., S. pneumonia and/or S. pyogenes). In some embodiments, the enhanced microbial strain inhibits and/or prevents the colonization of a substrate (e.g., human tissue) by one or more bacterial strains belonging to a species selected from the group consisting of Acinetobacter baumannii, Clostridium difficile, Enterococcus faecalis, Enterococcus faecium, Escherichia coli, Mycobacterium tuberculosis, Pseudomonas aeruginosa, Salmonella, Staphylococcus aureus, Staphylococcus epidermidis, Streptococcus pneumonia and Streptococcus pyogenes. In some embodiments, the enhanced microbial strain inhibits and/or prevents the colonization of a substrate (e.g., human tissue) by one or more bacterial strains that is least partially resistant to one or more antibiotics (e.g., clindamycin, erythromycin, isoniazid, linezolid, methicillin, penicillin, rifampin, streptomycin, tetracycline and/or vancomycin). In some embodiments, the enhanced microbial strain inhibits and/or prevents the colonization of a substrate (e.g., human tissue) by one or more bacterial strains selected from the group consisting of clindamycin-resistant Clostridium difficile, fluoroquinolone-resistant Clostridium difficile, fluoroquinolone-resistant Escherichia coli, fluoroquinolone-resistant Salmonella, isoniazid-resistant Mycobacterium tuberculosis, linezolid-resistant Enterococcus faecalis, linezolid-resistant Enterococcus facium, macrolide-resistant Streptococcus pyogenes, methicillin-resistant Staphylococcus aureus, methicilin-tesistant Staphylococcus epidermidis, multidrug-resistant Acinetobacter baumannii, multidrug-resistant Enterococcus faecalis, multidrug-resistant Enterococcus faecium, penicillin-resistant Streptococcus pneumoniae, penicillin-resistant Enterococcus faecalis, penicillin-resistant Enterococcus faecium, rifampin-resistant Mycobacterium tuberculosis, streptomycin-resistant Mycobacterium tuberculosis, vancomycin-resistant Enterococcus faecalis, vancomycin-resistant Enterococcus faecium, vancomycin-resistant Escherichia coli and vancomycin-resistant Staphylococcus aureus.

Enhanced microbial strains of the present invention may inhibit and/or prevent the colonization of a substrate (e.g., human tissue) by any suitable mold strain, including, but not limited to, pathogenic mold strains. In some embodiments, the enhanced microbial strain inhibits and/or prevents the colonization of a substrate (e.g., human tissue) by one or more mold strains belonging to a genus selected from the group consisting of Aspergillus (e.g., A. clavatus, A. fischerianus, A. flavus and/or A. fumigates), Histoplama (e.g., H. capsulatum), Pneumocystis (e.g., P. carinii and/or P. jirovecii) and Stachybotrys (e.g., S. charatum). In some embodiments, the enhanced microbial strain inhibits and/or prevents the colonization of a substrate (e.g., human tissue) by one or more mold strains belonging to a species selected from the group consisting of Aspergillus clavatus, Aspergillus fischerianus, Aspergillus flavus, Aspergillus fumigates, Histoplasma capsulatum, Pneumocystis carinii, Pneumocystis jirovecii and Stachybotrys chartarum.

Enhanced microbial strains of the present invention may inhibit and/or prevent the colonization of a substrate (e.g., human tissue) by any suitable yeast strain, including, but not limited to, pathogenic yeast strains. In some embodiments, the enhanced microbial strain inhibits and/or prevents the colonization of a substrate (e.g., human tissue) by one or more yeast strains belonging to a genus selected from the group consisting of Candida (e.g., C. albicans, C. glabrata, C. guilliermondi, C. krusei, C. lusitaniae, C. parapsilosis, C. stellatoidea, C. tropicalis and/or C. viswanathii), Cryptococcus (e.g., C. albidus, C. gattii, C. laurentii and/or C. neoformans), Rhodotorula (e.g., R. mucilaginosa), Torulopsis and Trichosporon. In some embodiments, the enhanced microbial strain inhibits and/or prevents the colonization of a substrate (e.g., human tissue) by one or more yeast strains belonging to a species selected from the group consisting of Candida albicans, Candida glabrata, Candida guilliermondi, Candida krusei, Candida lusitaniae, Candida parapsilosis, Candida stellatoidea, Candida tropicalis, Candida viswanathii, Cryptococcus albidus, Cryptococcus gattii, Cryptococcus laurentii, Cryptococcus neoformans and Rhodotorula mucilaginosa.

Enhanced microbial strains of the present invention may inhibit and/or prevent the proliferation of any suitable pathogenic microbial strain, including, but not limited to, bacterial strains, mold strains and yeast strains.

Enhanced microbial strains of the present invention may inhibit and/or prevent the proliferation of any suitable bacterial strain, including, but not limited to, pathogenic bacterial strains. In some embodiments, the enhanced microbial strain inhibits and/or prevents the proliferation of one or more bacterial strains belonging to a genus selected from the group consisting of Acinetobacter (e.g., A. baumannii), Clostridium (e.g., C. difficile), Enterococcus (e.g., E. faecalis and/or E. faecium), Escherichia (e.g., E. coli), Mycobacterium (e.g., M. tuberculosis), Pseudomonas (e.g., P. aeruginosa), Salmonella, Staphylococcus (e.g., S. aureus and/or S. epiderimis) and Streptococcus (e.g., S. pneumonia and/or S. pyogenes). In some embodiments, the enhanced microbial strain inhibits and/or prevents the proliferation of one or more bacterial strains belonging to a species selected from the group consisting of Acinetobacter baumannii, Clostridium difficile, Enterococcus faecalis, Enterococcus faecium, Escherichia coli, Mycobacterium tuberculosis, Pseudomonas aeruginosa, Salmonella, Staphylococcus aureus, Staphylococcus epidermidis, Streptococcus pneumonia and Streptococcus pyogenes. In some embodiments, the enhanced microbial strain inhibits and/or prevents the proliferation of one or more bacterial strains that is least partially resistant to one or more antibiotics (e.g., clindamycin, erythromycin, isoniazid, linezolid, methicillin, penicillin, rifampin, streptomycin, tetracycline and/or vancomycin). In some embodiments, the enhanced microbial strain inhibits and/or prevents the proliferation of one or more bacterial strains selected from the group consisting of clindamycin-resistant Clostridium difficile, fluoroquinolone-resistant Clostridium difficile, fluoroquinolone-resistant Escherichia coli, fluoroquinolone-resistant Salmonella, isoniazid-resistant Mycobacterium tuberculosis, linezolid-resistant Enterococcus faecalis, linezolid-resistant Enterococcus faecium, macrolide-resistant Streptococcus pyogenes, methicillin-resistant Staphylococcus aureus, methicilin-resistant Staphylococcus epidermidis, multidrug-resistant Acinetobacter baumannii, multidrug-resistant. Enterococcus faecalis, multidrug-resistant Enterococcus faecium, penicillin-resistant Streptococcus pneumoniae, penicillin-resistant Enterococcus faecalis, penicillin-resistant Enterococcus faecium, rifampin-resistant Mycobacterium tuberculosis, streptomycin-resistant Mycobacterium tuberculosis, vancomycin-resistant Enterococcus faecalis, vancomycin-resistant Enterococcus faecium, vancomycin-resistant Escherichia coil and vancomycin-resistant Staphylococcus aureus.

Enhanced microbial strains of the present invention may inhibit and/or prevent the proliferation of any suitable mold strain, including, but not limited to, pathogenic mold strains. In some embodiments, the enhanced microbial strain inhibits and/or prevents the proliferation of one or more mold strains belonging to a genus selected from the group consisting of Aspergillus (e.g., A. clavatus, A. fischerianus, A. flavus and/or A. fumigates), Histoplama (e.g., H. capsulatum), Pneumocystis (e.g., P. carinii and/or P. jirovecii) and Stachybotrys (e.g., S. charatum). In some embodiments, the enhanced microbial strain inhibits and/or prevents the proliferation of one or more mold strains belonging to a species selected from the group consisting of Aspergillus clavatus, Aspergillus fischerianus, Aspergillus flavus, Aspergillus fumigates, Histoplasma capsulatum, Pneumocystis carinii, Pneumocystis jirovecii and Stachybotrys chartarum.

Enhanced microbial strains of the present invention may inhibit and/or prevent the proliferation of any suitable yeast strain, including, but not limited to, pathogenic yeast strains. In some embodiments, the enhanced microbial strain inhibits and/or prevents the proliferation of one or more yeast strains belonging to a genus selected from the group consisting of Candida (e.g., C. albicans, C. glabrata, C. guilliermondi, C. krusei, C. lusitaniae, C. parapsilosis, C. stellatoidea, C tropicalis and/or C. viswanathii), Cryptococcus (e.g., C. albidus, C. gattii, C. laurentii and/or C. neoformans), Rhodotorula (e.g., R. mucilaginosa), Torulopsis and Trichosporon. In some embodiments, the enhanced microbial strain inhibits and/or prevents the proliferation of one or more yeast strains belonging to a species selected from the group consisting of Candida albicans, Candida glabrata, Candida guilliermondi, Candida krusei, Candida lusitaniae, Candida parapsilosis, Candida stellatoidea, Candida tropicalis, Candida viswanathii, Cryptococcus albidus, Cryptococcus gattii, Cryptococcus laurentii, Cryptococcus neoformans and Rhodotorula mucilaginosa.

Enhanced microbial strains of the present invention may kill any suitable pathogenic microbial strain, including, but not limited to, bacterial strains, mold strains and yeast strains.

Enhanced microbial strains of the present invention may inhibit kill any suitable bacterial strain, including, but not limited to, pathogenic bacterial strains. In some embodiments, the enhanced microbial strain kills one or more bacterial strains belonging to a genus selected from the group consisting of Acinetobacter (e.g., A. baumannii), Clostridium (e.g., C. difficile), Enterococcus (e.g., E. faecalis and/or E. faecium), Escherichia (e.g., E. coli), Mycobacterium (e.g., M. tuberculosis), Pseudomonas (e.g., P. aeruginosa), Salmonella, Staphylococcus (e.g., S. aureus and/or S. epiderimis) and Streptococcus (e.g., S. pneumonia and/or S. pyogenes). In some embodiments, the enhanced microbial strain kills one or more bacterial strains belonging to a species selected from the group consisting of Acinetobacter baumannii, Clostridium difficile, Enterococous faecalis, Enterococcus faecium, Escherichia coil, Mycobacterium tuberculosis, Pseudomonas aeruginosa, Salmonella, Staphylococcus aureus, Staphylococcus epidermidis, Streptococcus pneumonia and Streptococcus pyogenes. In some embodiments, the enhanced microbial strain kills one or more bacterial strains that is least partially resistant to one or more antibiotics (e.g., clindamyrin, erythromycin, isoniazid, linezolid, methicillin, penicillin, rifampin, streptomycin, tetracycline and/or vancomycin). In some embodiments, the enhanced microbial strain kills one or more bacterial strains selected from the group consisting of clindamycin-resistant Clostridium difficile, fluoroquinolone-resistant Clostridium difficile, fluoroquinolone-resistant Escherichia coli, fluoroquinolone-resistant Salmonella, isoniazid-resistant Mycobacterium tuberculosis, linezolid-resistant Enterococcus faecalis, linezolid-resistant Enterococcus faecium, macrolide-resistant Streptococcus pyogenes, methicillin-resistant Staphylococcus aureus, methicilin-resistant Staphylococcus epidermidis, multidrug-resistant Acinetobacter baumannii, multidrug-resistant Enterococcus faecalis, multidrug-resistant Enterococcus faecium, penicillin-resistant Streptococcus pneumoniae, penicillin-resistant Enterococcus faecalis, penicillin-resistant Enterococcus faecium, rifampin-resistant Mycobacterium tuberculosis, streptomycin-resistant Mycobacterium tuberculosis, vancomycin-resistant Enterococcus faecalis, vancomycin-resistant Enterococcus faecium, vancomycin-resistant Escherichia coli and vancomycin-resistant Staphylococcus aureus.

Enhanced microbial strains of the present invention may kill any suitable mold strain, including, but not limited to, pathogenic mold strains. In some embodiments, the enhanced microbial strain kills one or more mold strains belonging to a genus selected from the group consisting of Aspergillus (e.g., A. clavatus, A. fischerianus, A. flavus and/or A. fumigates), Histoplama (e.g., H. capsulatum), Pneumocystis (e.g., P. carinii and/or P. jirovecii) and Stachybotrys (e.g., S. charatum). In some embodiments, the enhanced microbial strain kills one or more mold strains belonging to a species selected from the group consisting of Aspergillus clavatus, Aspergillus fischerianus, Aspergillus flavus, Aspergillus fumigates, Histoplasma capsulatum, Pneumocystis carinii, Pneumocystis jirovecii and Stachybotrys chartarum.

Enhanced microbial strains of the present invention may kill any suitable yeast strain, including, but not limited to, pathogenic yeast strains. In some embodiments, the enhanced microbial strain kills one or more yeast strains belonging to a genus selected from the group consisting of Candida (e.g., C. albicans, C. glabrata, C. guilliermondi, C. krusei, C. lusitaniae, C. parapsilosis, C. stellatoidea, C. tropicalis and/or C. viswanathii), Cryptococcus (e.g., C. albidus, C. gattii, C. laurentii and/or C. neoformans), Rhodotorula (e.g., R. mucilaginosa), Torulopsis and Trichosporon. In some embodiments, the enhanced microbial strain kills one or more yeast strains belonging to a species selected from the group consisting of Candida albicans, Candida glabrata, Candida guilliermondi, Candida krusei, Candida lusitaniae, Candida parapsilosis, Candida stellatoidea, Candida tropicalis, Candida viswanathii, Cryptococcus albidus, Cryptococcus gattii, Cryptococcus laurentii, Cryptococcus neoformans and Rhodotorula mucilaginosa.

Enhanced microbial strains of the present invention may produce any suitable inhibitory substance, including, but not limited to, alcohols, antibiotics, beta lactams, biofilm disruption molecules, carbon dioxide, growth inhibitors and toxic peptides. In some embodiments, the enhanced microbial strain produces one or more inhibitory substances in greater amounts than does the microbial strain from which it was derived. For example, the enhanced microbial strain may produce an amount of inhibitory substance that is at least about 5%, 10%, 15%, 20%, 25%, 30%, 35%, 40%, 45%, 50%, 55%, 60%, 65%, 70%, 75%, 80% 85%, 95% 100%, 125%, 150%, 175%, 200%, 250%, 300%, 350%, 400%, 450%, 500%, 600%, 700%, 800%, 900% or more greater than the amount produced by the microbial strain from which it was derived. In some embodiments, the enhanced microbial strain produces one or more inhibitory substances at a greater rate than does the microbial strain from which it was derived. For example, the enhanced microbial strain may produce an inhibitory substance at a rate that is at least about 5%, 10%, 1 5%, 20%, 25%, 30%, 35%, 40%, 45%, 50%, 55%, 60%, 65%, 70%, 75%, 80% 85%, 95% 100%, 125%, 150%, 175%, 200%, 250%, 300%, 350%, 400%, 450%, 500%, 600%, 700%, 800%, 900% or more higher than the rate at which the microbial strain from which it was derived produces that same inhibitory substance. In some embodiments, the enhanced microbial strain releases one or more inhibitory substances into its environment at a greater rate than does the microbial strain from which it was derived. For example, the enhanced microbial strain may release an inhibitory substance at a rate that is at least about 5%, 10%, 15%, 20%, 25%, 30%, 35%, 40%, 45%, 50%, 55%, 60%, 65%, 70%, 75%, 80% 85%, 95% 100%, 125%, 150%, 175%, 200%, 250%, 300%, 350%, 400%, 450%, 500%, 600%, 700%, 800%, 900% or more higher than the rate at which the microbial strain from which it was derived releases that same inhibitory substance.

Enhanced microbial strains of the present invention may raise the pH of their environments via any suitable means, including, but not limited to, releasing calcium and/or magnesium into the environment. In some embodiments, the enhanced microbial strain raises the pH of its environment by changing the concentration of one or more ions in the environment (e.g., by increasing and/or decreasing release of one or more ions, by increasing and/or decreasing sequestration of one or more ions and/or by increased lysosomal activity). In some embodiments, the enhanced microbial strain raises the pH of its environment to a greater extent than does the microbial strain from which it was derived. For example, the enhanced microbial strain may raise the pH of its environment by at least about 5%, 10%, 15%, 20%, 25%, 30%, 35%, 40%, 45%, 50%, 55%, 60%, 65%, 70%, 75%, 80% 85%, 95% 100%, 125%, 150%, 175%, 200%, 250%, 300%, 350%, 400%, 450% or 500% more than the microbial strain from which it was derived raises the pH of its environment. In sonic embodiments, the enhanced microbial strain raises the pH of its environment at a greater rate than does the microbial strain from which it was derived. For example, the enhanced microbial strain may raise the pH of its environment at a rate that is at least about 5%, 10%, 15%, 20%, 25%, 30%, 35%, 40%, 45%, 50%, 55%, 60%, 65%, 70%, 75%, 80% 85%, 95% 100%, 125%, 150%, 175%, 200%, 250%, 300%, 350%, 400%, 450%, 500%, 600%, 700%, 800%, 900% or more higher than the rate at which the microbial strain from which it was derived raises the of its environment.

Enhanced microbial strains of the present invention may lower the pH of their environments via any suitable means, including, but not limited to, releasing carbon dioxide and/or one or more organic acids (e.g., acetic acid, benzoic acid, chloroacetic acid, citric acid, dichloroacetic acid, formic acid, lactic acid, oxalic acid, taurine, triehloroacetic acid, trifluoracetie and/or uric acid) into the environment. In some embodiments, the enhanced microbial strain lowers the of its environment by changing the concentration of one or more ions in the environment (e.g., by increasing and/or decreasing release of one or more ions, by increasing and/or decreasing sequestration of one or more ions and/or by decreased lysosomal activity). In some embodiments. the enhanced microbial strain lowers the pH of its environment to a greater extent than does the microbial strain from which it was derived. For example, the enhanced microbial strain may lower the pH of its environment by at least about 5%, 10%, 15%, 20%, 25%, 30%, 35%, 40%, 45%, 50%, 55%, 60%, 65%, 70%, 75%, 80% 85%, 95% 100%, 125%, 150%, 175%, 200%, 250%, 300%, 350%, 400%, 450% or 500% more than the microbial strain from which it was derived lowers the pH of its environment. In some embodiments, the enhanced microbial strain lowers the pH of its environment at a greater rate than does the microbial strain from which it was derived. For example, the enhanced microbial strain may lower the pH of its environment at a rate that is at least about 5%, 10%, 15%, 20%, 25%, 30%, 35%, 40%, 45%, 50%, 55%, 60%, 65%, 70%, 75%, 80% 85%, 95% 100%, 125%, 150%, 175%, 200%, 250%, 300%, 350%, 400%, 450%, 500%, 600%, 700%, 800%, 900% or more higher than the rate at which the microbial strain from which it was derived lowers the pH of its environment.

Enhanced microbial strains of the present invention may raise the alkalinity of their environments via any suitable means, including, but not limited to, releasing calcium and/or magnesium into the environment. In some embodiments, the enhanced microbial strain raises the to of its environment by changing the concentration of one or more ions in the environment (e.g., by increasing and/or decreasing release of one or more ions, by increasing and/or decreasing sequestration of one or more ions and/or by increased lysosomal activity). In some embodiments, the enhanced microbial strain raises the pH of its environment to a greater extent than does the microbial strain from which it was derived. For example, the enhanced microbial strain may raise the alkalinity of its environment by at least about 5%, 10%, 15%, 20%, 25%, 30%, 35%, 40%, 45%, 50%, 55%, 60%, 65%, 70%, 75%, 80% 85%, 95% 100%, 125%, 150%, 175%, 200%, 250%, 300%, 350%, 400%, 450% or 500% more than the microbial strain from which it was derived raises the alkalinity of its environment.

In some embodiments, the enhanced microbial strain raises the alkalinity of its environment at a greater rate than does the microbial strain from which it was derived. For example, the enhanced microbial strain may raise the alkalinity of its environment at a rate that is at least about 5%, 10%, 15%, 20%, 25%, 30%, 35%, 40%, 45%, 50%, 55%, 60%, 65%, 70%, 75%, 80% 85%, 95% 100%, 125%, 150%, 175%, 200%, 250%, 300%, 350%, 400%, 450%, 500%, 600%, 700%, 800%, 900% or more higher than the rate at which the microbial strain from which it was derived raises the alkalinity of its environment.

Enhanced microbial strains of the present invention may lower the alkalinity of their environments via any suitable means, including, but not limited to, releasing carbon dioxide and/or one or more organic acids (e.g., acetic acid, benzoic acid, chloroacetic acid, citric acid, dichloroacetic acid, formic acid, lactic acid, oxalic acid, taurine, trichloroacetic acid, trifluoracetic and/or uric acid) into the environment. In some embodiments, the enhanced microbial strain lowers the alkalinity of its environment by changing the concentration of one or more ions in the environment (e.g., by increasing and/or decreasing release of one or more ions, by increasing and/or decreasing sequestration of one or more ions and/or by decreased lysosomal activity). In some embodiments, the enhanced microbial strain lowers the alkalinity of its environment to a greater extent than does the microbial strain from which it was derived. For example, the enhanced microbial strain may lower the alkalinity of its environment by at least about 5%, 10%, 15%, 20%, 25%, 30%, 35%, 40%, 45%, 50%, 55%, 60%, 65%, 70%, 75%, 80% 85%, 95% 100%, 125%, 150%, 175%, 200%, 250%, 300%, 350%, 400%, 450% or 500% more than the microbial strain from which it was derived lowers the alkalinity of its environment. In some embodiments, the enhanced microbial strain lowers the alkalinity of its environment at a greater rate than does the microbial strain from which it was derived. For example, the enhanced microbial strain may lower the alkalinity of its environment at a rate that is at least about 5%, 10%, 15%, 20%, 25%, 30%, 35%, 40%, 45%, 50%, 55%, 60%, 65%, 70%, 75%, 80% 85%, 95% 100%, 125%, 150%, 175%, 200%, 250%, 300%, 350%, 400%, 450%, 500%, 600%, 700%, 800%, 900% or more higher than the rate at which the microbial strain from which it was derived lowers the alkalinity of its environment.

Enhanced microbial strains of the present invention may raise the level of one or more gases in their environments via any suitable means, including, but not limited to, releasing carbon dioxide, methane and/or oxygen into the environment. In some embodiments, the enhanced microbial strain raises the level of one or more gases in its environment to a greater extent than does the microbial strain from which it was derived. For example, the enhanced microbial strain may raise the alkalinity of its environment by at least about 5%, 10%, 15%, 20%, 25%, 30%, 35%, 40%, 45%, 50%, 55%, 60%, 65%, 70%, 75%, 80% 85%, 95% 100%, 125%, 150%, 175%, 200%, 250%, 300%, 350%, 400%, 450% or 500% more than the microbial strain from which it was derived raises the level of one or more gases in its environment. In some embodiments, the enhanced microbial strain raises the level of one or more gases in its environment at a greater rate than does the microbial strain from which it was derived. For example, the enhanced microbial strain may raise the level of one or more gases in its environment at a rate that is at least about 5%, 10%, 15%, 20%, 25%, 30%, 35%, 40%, 45%, 50%, 55%, 60%, 65%, 70%, 75%, 80% 85%, 95% 100%, 125%, 150%, 175%, 200%, 250%, 300%, 350%, 400%, 450%, 500%, 600%, 700%, 800%, 900% or more higher than the rate at which the microbial strain from which it was derived raises the level of one or more gases in its environment.

Enhanced microbial strains of the present invention may lower the level of one or more gases in their environments via any suitable means, including, but not limited to, sequestering carbon dioxide, methane and/or oxygen into the environment. In some embodiments, the enhanced microbial strain lowers the level of one or more gases in its environment to a greater extent than does the microbial strain from which it was derived. For example, the enhanced microbial strain may lower the alkalinity of its environment by at least about 5%, 10%, 15%, 20%, 25%, 30%, 35%, 40%, 45%, 50%, 55%, 60%, 65%, 70%, 75%, 80% 85%, 95% 100%, 125%, 150%, 175%, 200%, 250%, 300%, 350%, 400%, 450% or 500% more than the microbial strain from which it was derived lowers the level of one or more gases in its environment. In some embodiments, the enhanced microbial strain lowers the level of one or more gases in its environment at a greater rate than does the microbial strain from which it was derived. For example, the enhanced microbial strain may lower the level of one or more gases in its environment at a rate that is at least about 5%, 10%, 15%, 20%, 25%, 30%, 35%, 40%, 45%, 50%, 55%, 60%, 65%, 70%, 75%, 80% 85%, 95% 100%, 125%, 150%, 175%, 200%, 250%, 300%, 350%, 400%, 450%, 500%, 600%, 700%, 800%, 900% or more higher than the rate at which the microbial strain from which it was derived lowers the level of one or more gases in its environment.

Enhanced microbial strains of the present invention may promote the colonization of a substrate (e.g., human tissue) by any suitable microbial strain, including, but not limited to, bacterial strains, mold strains and yeast strains.

Enhanced microbial strains of the present invention may promote the colonization of a substrate (e.g. human tissue) by any suitable bacterial strain, including, but not limited to, probiotic bacterial strains and commensal bacterial strains. In some embodiments, the enhanced microbial strain promotes colonization of the substrate by one or more bacterial strains that produce lactic acid. In some embodiments, the enhanced microbial strain promotes colonization of the substrate by one or more bacterial strains belonging to a genus selected from the group consisting of Carnobacterium (e.g., C. alterfunditum, C. divergens C. funditum, C. gallinarium, C. inhibens, C. jeotgali, C. maltaromaticum, C. mobile, C. piscicola, C. pleistocenium and/or C. viridans), Enterococcus (e.g., E. aquimarinus, E. asini, E. avium, E. caccae, E. camelliae, E. canintestini, E. canis, E. casseloflavus, E. cecorum, E. columbae, E. devriesei, E. dispar, E. durans, E. faecalis, E. facium, E. flavescens, E. gallnarum, E. gilvus, E. haemoperoxidus, E. hermanniensis, E. hirae, E. iatlicus, E. lactis, E. maodoratus, E. moraviensis, E. mundatii, E. pallens, E. phoeniculicola, E. plantarum, E. porcinus, E. pseudoavium, E. quebecensis, E. raffinosus, E. ratii, E. rivorum, E. saccharolyticus, E. sacchoromimimus, E. seriolicida, E. silesiacus, E. solitarius, E. sulfureus, E. termitis, E. thailandicus, E. ureasiticus, E. vikkiensis and/or E. villorum), Lactobacillus (e.g., L. acetotolerans, L. acidifarinae, L. acidipiscis, L. acidophilus, L. agilis, L. algidus, L. alimentarius, L. amylolyticus, L. amylophilus, L. amylotrophicus, L. amylovorus, L. animalis, L. antri, L. apodemi, L. aquaticus, L. arizonensis, L. aviarius, L. bavaricus, L. bifermentans, L. bobalius, L. brantae, L. brevis, L. buchneri, L. bulgarius, L. caaonum, L. camelliae, L. capillatus, L. carnis, L. casei, L. catenaformis, L. cellobiosus, L. ceti, L. coleohominis, L. collinoides, L. composti, L. concavus, L. confusus, L. coryniformis, L. crispatus, L. crustorum, L. curvatus, L. cypricasei, L. delbrueckii, L. dextrinicus, L. diolovorans, L. divergens, L. durianis, L. equi, L. equicursoris, L. equigenerosi, L. fabifermentans, L. farciminis, L. farraginis, L. ferintoshensis, L. fermentum, L. floricola, L. florum, L. fronicalis, L. fructivorans, L. fructosus, L. frumenti, L. fuchensis, L. futsaii, L. gallinarium, L. gasseri, L. gastricus, L. ghanensis, L. gigeriorum, L. graminis, L. halotolerans, L. hammesii, L. hamsteri, L. harbinensis, L. hayakitensis, L. helveticus, L. heterochii, L. hilgardii, L. hominis, L. homohiochii, L. hordei, L. iners, L. ingluviei, L. intestinalis, L. jensenii, L. johnsonii, L. kalixensis, L. kandleri, L. kerfiranofaciens, L. kefirgranum, L. kefiri, L. kimchicus, L. kimchii, L. kisonensis, L. kitasatonis, L. koreensis, L. kunkeei, L. lactis, L. leichmannii, L. lindneri, L. malefermentans, L. mali, L. maltaromicus, L. manihotivorans, L. mindensis, L. minor, L. minutus, L. mucosae, L. murinus, L. nagelii, L. namurensis, L. nantensis, L. nasuensis, L. nodensis, L. odoratitofui, L. oeni, L. oligafermentans, L. oris, L. otakiensis, L. ozensis, L. panis, L. pantheris, L. parabrevis, L. parabuchneri, L. paracasei, L. paracollinoides, L. parafarraginis, L. parakefiri, L. paralimentarius, L. paraplantarum, L. pasteurii, L. paucivorans, L. pentosus, L. perolens, L. piscicola, L. plantarum, L. pobuzihii, L. pontis, L. psittaci, L. rapi, L. rennini, L. reuteri, L. rhamnosus, L. rimae, L. rogosae, L. rossiae, L. ruminis, L. saerimneri, L. sakei, L. salivarius, L. sanfranciscenis, L. saniviri, L. satsumensis, L. secaliphilus, L. selangorensis, L. senioris, L. senmaizukei, L. sharpeae, L. siliginis, L. similis, L. sobrius, L. spicheri, L. sporogenes, L. sucicola, L. suebicus, L. sunkii, L. suntoryeus, L. taiwanensis, L. thailandensis, L. thermotolerans, L. trchodes, L. tucceti, L. uli, L. ultunensis, L. uvarium, L. vaccinostercus, L. vaginalis, L. versmoldensis, L. vini, L. viridescens, L. vitulinus, L. xiangfangensis, L. xylosus, L. yamanashiensis, L. zeae and/or L. zymae), Lactococcus (e.g., L. chugangensis, L. fujiensis, L. garvieae, L. lactis, L. piscium, L. plantarum and/or L. raffinolactis), Leuconostoc (e.g., L. amelibiosum. L. argentinum, L. carnosum, L. citreum, L. cremoris, L. dextranicum, L. durionis, L. fallux, L. ficulmeum, L. fructosum, L. gasicomitatum, L. geidum, L. holzapfelii, L. inhae, L. kimchii, L. lactis, L. mesenteroides, L. miyukkimchii, L. oeni, L. palmae, L. paramesenteroides, L. pseudoficulneum and/or L. pseudomesenteroides), Oenococeus (e.g., O. kitaharae and/or O. oeni), Pediococcus (e.g., P. argentinicus, P. cellicola, P. claussenii, P. damnosus, P. dextrinicus, P. ethanolidruans, P. halophilus, P. inopinatus, P. lolii, P. parvulus, P. pentosaceus, P. siamensis, P. stilesii and/or P. urinaeequi) Streptococcus (e.g., S. acidominimus, S adjacens, S. agalactiae, S. alactolyticus, S. anginosus, S. australius, S. bovis, S. caballi, S. canis, S. caprinus, S. castoreus, S. cecorum, S. constellatus, S. cremoris, S. criceti, S. cristatus, S. defectivus, S. dentapri, S. dentirousetti, S. devriesei, S. didelphis, S. difficilis, S. downei, S. durans, S. dysgalactiae, S. entericus, S. equi, S. equinus, S. faecalis, S. faecium, S. ferus, S. gallinaceus, S. gallnarium, S. gallolyticus, S. garvieae, S. gordonii, S. halichoeri, S. hansenii, S. henryi, S. hyointestinalis, S. hyovaginalis, S. ictaluri, S. infantarius, S. infantis, S. iniae, S. intermedius, S. intestinalis, S. lactarius, S. lactis, S. lutetiensis, S. macaccae, S. macedonicus, S. marimammalium, S. massiliensis, S. merionis, S. minor, S. mitis S. morbillorum, S. mutans, S. oligofermentans, S. oralis, S. orisratti, S. orisuis, S. ovis, S. parasanguinis, S. parauberis, S. paravuluvs, S. pasteurianus, S. peroris, S. phocae, S. plantarum, S. pleomorphus, S. pluranimalium, S. plurextorum, S. pneumoniae, S. porci, S. porcinus, S. porcorum, S. pseudopneumoniae, S. pseudoporcinus, S. pyogenes, S. raffinolactis, S. ratii, S. rupicaprae, S. saccharolyticus, S. salivarius, S. sanguinis, S. shiloi, S. sinensis, S. sobrinus, S. suis, S. thermophilus, S. thoraltensis, S. tigurinus, S. uberis, S. urinalis, S. ursoris, S. vestibularis and/or S. waius), Tetragenococcus (e.g., T. halophilus, T. koreensis, T. muriaticus, T. osmophilus and/or T. solitarius), Vagococcus (e.g., V. acidifermentans, V. carniphilus, V elongatus, V. fessus, V. fluvialis, V. lutrae, V. penaei and/or V. salmoninarum) and Weissella (e.g., W. beninensis, W. ceti, W. cibaria, W. confusa, W. fabaria, W. ghanensis, W. halotolerans, W. hellenica, W. kandleri, W. kimchii, W. koreensis, W. minor, W. paramesenteroides, W. soli, W. thailandensis and/or W. viridescens). In some embodiments, the enhanced microbial strain promotes colonization of the substrate by one or more bacterial strains belonging to a species selected from the group consisting of Lactobacillus acidophilus, Lactobacillus brevis, Lactobacillus buchneri, Lactobacillus casei, Lactobacillus curvatus, Lactobacillus delbrueckii, Lactobacillus fermentum, Lactobacillus helveticus, Lactobacillus plantarum, Lactobacillus reuteri, Lactobacillus sakei, Lactobacillus sporogenes and Lactobacillus salivarius.

Enhanced microbial strains of the present invention may promote the colonization of a substrate (e.g., human tissue) by any suitable mold strain, including, but not limited to, probiotic mold strains and commensal mold strains.

Enhanced microbial strains of the present invention may promote the colonization of a substrate (e.g., human tissue) by any suitable yeast strain, including, but not limited to, probiotic yeast strains and commensal yeast strains. In some embodiments, the enhanced microbial strain promotes colonization of the substrate by one or more yeast strains belonging to a genus selected from the group consisting of Brettanomyces (e.g., B. anomalus, B. bruxellensis, B. custersianus, B. naardenensis and/or B. nanus), Candida (e.g., C. stellata), Dekkera (e.g., D. anomala and/or D. bruxellensis), Saccharomyces (e.g., S. cerevisiae, S. bayanus, S. boulardii and/or S. pastorianus), Schizosaccharomyces (e.g., S. pombe), Torulaspora (e.g., T. delbrueckii), Torulopsis and Zygosaccharomyces (e.g., Z. bailii). In some embodiments, the enhanced microbial strain promotes colonization of the substrate by one or more yeast strains belonging to a species selected from the group consisting of Brettanomyces anomalus, Brettanomyces bruxellensis, Brettanomyces custersianus, Brettanomyces naardenensis, Brettanomycesnanus, Dekkera anomala, Dekkera bruxellensis, Saccharomyces cerevisiae, Saccharomyces bayanus, Saccharomyces boulardii and Saccharomyces pastorianus.

Enhanced microbial strains of the present invention may promote the colonization of a substrate (e.g., human tissue) by one or more other microbial strains (e.g., one or more probiotic microbial strains) via any suitable means, including, but not limited to, the production of one or more substances that promote the colonization of the substrate by the other microbial strain(s) biofilm constituents such as glycolipids, glycoproteins and/or proteoglycans). In some embodiments, the enhanced microbial strain produces more of a substance (or substances) that promote(s) the colonization of the substrate by the other microbial strain(s) than does the microbial strain from which it was derived. For example, the enhanced microbial strain may produce at least about 5%, 10%, 1 5%, 20%, 25%, 30%, 35%, 40%, 45%, 50%, 55%, 60%, 65%, 70%, 75%, 80% 85%, 95% 100%, 125%, 150%, 175%, 200%, 250%, 300%, 350%, 400%, 450% or 500% more of the substance(s) than the microbial strain from which it was derived. In some embodiments, the enhanced microbial strain produces one or more substances that promote the colonization of the substrate by the other microbial strain(s) at a greater rate than does the microbial strain from which it was derived. For example, the enhanced microbial strain may produce a substance that promotes the colonization of the substrate by the other microbial strain(s) at a rate that is at least about 5%, 10%, 15%, 20%, 25%, 30%, 35%, 40%, 45%, 50%, 55%, 60%, 65%, 70%, 75%, 80% 85%, 95% 100%, 125%, 150%, 175%, 200%, 250%, 300%, 350%, 400%, 450%, 500%, 600%, 700%, 800%, 900% or more higher than the rate at which the microbial strain from which it was derived produces that same substance. In some embodiments, the enhanced microbial strain releases one or more substances that promote the colonization of the substrate by the other microbial strain(s) into its environment at a greater rate than does the microbial strain from which it was derived. For example, the enhanced microbial strain may release a substance that promotes the colonization of the substrate by the other microbial strain(s) at a rate that is at least about 5%, 10%, 15%, 20%, 25%, 30%, 35%, 40%, 45%, 50%, 55%, 60%, 65%, 70%, 75%, 80% 85%, 95% 100%, 125%, 150%, 175%, 200%, 250%, 300%, 350%, 400%, 450%, 500%, 600%, 700%, 800%, 900% or more higher than the rate at which the microbial strain from which it was derived releases that same substance.

Enhanced microbial strains of the present invention may promote the growth and/or proliferation of any suitable microbial strain, including, but not limited to, bacterial strains, mold strains and yeast strains.

Enhanced microbial strains of the present invention may promote the growth and/or proliferation of any suitable bacterial strain, including, but not limited to, probiotic bacterial strains and commensal bacterial strains. In some embodiments, the other microbial strain is derived from a bacterial strain that produces lactic acid. In some embodiments, the other microbial strain is derived from a bacterial strain belonging to a genus selected from the group consisting of Carnobacterium (e.g., C. alterfunditum, C. divergens C funditum, C. gallinarium, C. inhibens, C jeotgali, C. maltaromaticum, C. mobile, C. piscicola, C. pleistocenium and/or C. viridans), Enterococcus (e.g., E. aquimarinus, E. asni, E. avium, E. caccae, E. camelliae, E. canintestini, E. canis, E. casseloflavus, E. cecorum, E. columbae, E. devriesei, E. dispar, E. durans, E. faecalis, E. faecium, E. flavescens, E. gallnarum, E. gilvus, E. haemoperoxidus, E. hermanniensis, E. hirae, E. iatlicus, E. lactis, E. maodoratus, E. moraviensis, F. mundatii, E. pallens, E. phoeniculicola, E. plantarum, E. porcinus, E. pseudoavium, E. quebecensis, E. raffinosus, E. ratti, E. rivorum, E. saccharolyticus, E. saccharomimimus, E. seriolicida, E. silesiacus, E. solitarius, E. sulfureus, E. termitis, E. thailandicus, E. ureasiticus, E. vikkiensis and/or E. villorum) Lactobacillus (e.g., L. acetotolerans, L. acidifarinae, L. acidipiscis, L. acidophilus, L. agilis, L. algidus, L. alimentarius, L. amylolyticus, L. amylophilus, L. amylatrophicus, L. amylovorus, L. animalis, L. antri, L. apodemi, L. aquaticus, L. arizonensis, L. aviarius, L. bavaricus, L. bifermentans, L. bobalius, L. brantae, L. brevis, L. buchneri, L. bulgarius, L. caaonum, L. camelliae, L. capillatus, L. carnis, L. casei, L. catenaformis, L. cellobiosus, L. ceti, L. coleohominis, L. collinoides, L. composti, L. concavus, L. confusus, L. coryniformis, L. crispatus, L. crustorum, L. curvatus, L. cypricasei, L. delbrueckii, L. dextrinicus, L. diolovorans, L. divergens, L. durianis, L. equi, L. equicursoris, L. equigenerosi, L. fabifermentans, L. farciminis, L. farraginis, L. ferintoshensis, L. fermentum, L. floricola, L. florum, L. fronicalis, L. fructivorans, L. fructosus, L. frumenti, L. fuchuensis, L. futsaii, L. gallinarium, L. gasseri, L. gastricus, L. ghanensis, L. gigeriorum, L. graminis, L. halotolerans, L. hammesii, L. hamsteri, L. harbinensis, L. hayakitensis, L. helveticus, L. heterochii, L. hilgardii, L. hominis, L. homohiochii, L. hordei, L. iners, L. ingluviei, L. intestinalis, L. jensenii, L. johnsonii, L. kalixensis, L. kandleri, L. kerfiranofaciens, L. kefirgranum, L. kefiri, L. kimchicus, L. kimchii, L. kisonensis, L. kitasatonis, L. koreensis, L. kunkeei, L. lactis, L. leichmannii, L. lindneri, L. malefermentans, L. mali, L. maltaromicus, L. manihotivorans, L. mindensis, L. minor, L. minutus, L. mucosae, L. murinus, L. nagelii, L. namurensis, L. nantensis, L. nasuensis, L. nodensis, L. odoratitofui, L. oeni, L. oligofermentans, L. oris, L. otakiensis, L. ozensis, L. panis, L. pantheris, L. parabrevis, L. parabuchneri, L. paracasei, L. paracollinoides, L. parafarraginis, L. parakefiri, L. paralimentarius, L. paraplantarum, L. pasteurii, L. paucivorans, L. pentosus, L. perolens, L. piscicola, L. plantarum, L. pobuzihii, L. pontis, L. psittaci, L. rapi, L. rennini, L. reuteri, L. rhamnosus, L. rimae, L. rogosae, L. rossiae, L. ruminis, L. saerimneri, L. sakei, L. salivarius, L. sanfranciscenis, L. saniviri, L. satsumensis, L. secaliphilus, L. selangorensis, L. senioris, L. senmaizukei, L. sharpeae, L. siliginis, L. similis, L. sobrius, L. spicheri, L. sporogenes, L. sucicola, L. suebicus, L. sunkii, L. suntoryeus, L. taiwanensis, L. thailandensis, L. thermotolerans, L. trchodes, L. tucceti, L. uli, L. ultunensis, L. uvarium, L. vaccinostercus, L. vaginalis, L. versmoldensis, L. vini, L. viridescens, L. vitulinus, L. xiangfangensis, L. xylosus, L. yamanashiensis, L. zeae and/or L. zymae), Lactococcus (e.g., L. chugangensis, L. fujiensis, L. garvieae, L. lactis, L. piscium, L. plantarum and/or L. raffinolactis), Leuconostoc (e.g., L. amelibiosum, L. argentinum, L. carnosum, L. citreum, L. cremoris, L. dextranicum, L. durionis, L. fallax, L. ficulneum, L. fructosum, L. gasicomitatum, L. geidum, L. holzapfelii, L. inhae, L. kimchii, L. lactis, L. mesenteroides, L. miyukkimchii, L. oeni, L. palmae, L. paramesenteroides, L. pseudoficulneum and/or L. pseudomesenteroides), Oenococcus (e.g., O. kitaharae and/or O. oeni), Pediococcus (e.g., P. argentinicus, P. cellicola, P. claussenii, P. damnosus, P. dextrinicus, P. ethanolidruans, P. halophilus, P. inopinatus, P. lolii, P. parvulus, P. pentosaceus, P. siamensis, P. stilesii and/or P. urinaeequi) Streptococcus (e.g., S. acidominimus, S. adjacens, S. agalactiae, S. alactolyticus, S. anginosus, S. australius, S. bovis, S. caballi, S. canis, S. caprinus, S. castoreus, S. cecorum, S. constellatus, S. cremoris, S. criceti, S. cristatus, S. defectivus, S. dentapri, S. dentirousetti, S. devriesei, S. didelphis, S. difficilis, S. downei, S. durans, S. dysgalactiae, S. entericus, S. equi, S. equinus, S. faecalis, S. faecium, L. ferus, S. gallinaceus, S. gallnarium, S. gallolyticus, S. garvieae, S. gordonii, S. halichoeri, S. hansenii, S. henryi, S. hyointestinalis, S. hyovaginalis, S. ictaluri, S. infantarius, S. infantis, S. iniae, S. intermedius, S. intestinalis, S. lactarius, S. lactis, S. lutetiensis, S. macacae, S. macedonicus, S. marimammalium, S. massiliensis, S. merionis, S. minor, S. mitis, S. morbillorum, S. mutans, S. oligofermentans, S. oralis, S. orisratti, S. orisuis, S. ovis, S. parasanguinis, S. parauberis, S. paravuluvs, S. pasteurianus, S. peroris, S. phocae, S. plantarum, S. pleomorphus, S. pluranimalium, S. plurextorum, S. pneumoniae, S. porci, S. porcinus, S. porcorum, S. pseudopneumoniae, S. pseudoporcinus, S. pyogenes, S. raffinolactis, S. ratti, S. rupicaprae, S. saccharolyticus, S. salivarius, S sanguinis, S. shiloi, S. sinensis, S. sobrinus, S. suis, S. thermophilus, S. thoraltensis, S. tigurinus, S. uberis, S. urinalis, S. ursoris, S. vestibularis and/or S. waius), Tetragenococcus (e.g., T. halophilus, T. koreensis, T. muriaticus, t. osmophilus and/or S. solitarius), Vagococcus (e.g., V. acidifermentans, V. carniphilus, V. elongotus, V. fessus, VV. fluvialis, V. lurae, V. penaei and/or V. salmoninarum) and Weissella (e.g., W. beninensis, W. ceti, W. cibaria, W confusa, W. fabaria, W. ghanensis, W. halotolerans, W. hellenica, W. kandleri, W. kimchii, W. koreensis, W. minor, W. paramesenteoides, W. soli, W. thailandensis and/or W. viridescens). In some embodiments, the other microbial strain is derived from a bacterial strain belonging to a species selected from the group consisting of Lactobacillus acidophilus, Lactobacillus brevis, Lactobacillus buchneri, Lactobacillus casei, Lactobacillus curvatus, Lactobacillus delbrueckii, Lactobacillus fermentum, Lactobacillus helveticus, Lactobacillus plantarum, Lactobacillus reuteri, Lactobacillus sakei, Lactobacillus sporogenes and Lactobacillus sallvarius.

Enhanced microbial strains of the present invention may promote the growth and/or proliferation of any suitable mold strain, including, but not limited to, probiotic mold strains and commensal mold strains.

Enhanced microbial strains of the present invention may promote the growth and/or proliferation of any suitable yeast strain, including, but not limited to, probiotic yeast strains and commensal yeast strains. In some embodiments, the other microbial strain is derived from a yeast strain belonging to a genus selected from the group consisting of Brettanomyces (e.g., B. anomalus, B. bruxellensis, B. custersianus, B. naardenensis and/or B. nanus), Candida (e.g., C. stellata), Dekkera (e.g., D. anomala and/or D. bruxellensis), Saccharomyces (e.g., S. cerevisiae, S. bayanus, S. boulardii and/or S. pastorianus), Schizosaccharomyces (e.g., S. pombe), Torulaspora (e.g., T. delbrueckii), Torulopsis and Zygosaccharomyces (e.g., Z. bailii). In some embodiments, the other microbial strain is derived from a yeast strain belonging to a species selected from the group consisting of Brettanomyces anomalus, Brettanomyces bruxellensis, Brettanomyces custersianus, Brettanomyces naardenensis, Brettanomycesnanus, Dekkera anomala, Dekkera bruxellensis, Saccharomyces cerevisiae, Saccharomyces boyanus, Saccharomyces boulardii and Saccharomyces pastorianus.

Enhanced microbial strains of the present invention may promote the growth and/or proliferation of one or more other microbial strains (e.g., one or more probiotic microbial strains) via any suitable means, including, but not limited to, the production of one or more substances that promote the growth and/or proliferation of the other microbial strain(s)). in some embodiments, the other microbial strain(s) grow and/or proliferate at a greater rate in the presence of the enhanced microbial strain than in the presence of the microbial strain from which the enhanced microbial strain was derived. For example, the growth and/or proliferation rates of the other microbial strain(s) may be at least about 5%, 10%, 15%, 20%, 25%, 30%, 35%, 40%, 45%, 50%, 55%, 60%, 65%, 70%, 75%, 80% 85%, 95% 100%, 125%, 150%, 175%, 200%, 250%, 300%, 350%, 400%, 450%, 500%, 600%, 700%, 800%, 900% or more higher in the presence of the enhanced microbial strain than in the presence of the microbial strain from which the enhanced microbial strain was derived. In some embodiments, the enhanced microbial strain produces more of a substance (or substances) that promote(s) the growth and/or proliferation of the other microbial strain(s) than does the microbial strain from which it was derived. For example, the enhanced microbial strain may produce at least about 5%, 10%, 15%, 20%, 25%, 30%, 35%, 40%, 45%, 50%, 55%, 60%, 65%, 70%, 75%, 80% 85%, 95% 100%, 125%, 150%, 175%, 200%, 250%, 300%, 350%, 400%, 450% or 500% more of the substance(s) than the microbial strain from which it was derived. In some embodiments, the enhanced microbial strain produces one or more substances that promote the growth and/or proliferation of the other microbial strain(s) at a greater rate than does the microbial strain from which it was derived. For example, the enhanced microbial strain may produce a substance that promotes the growth and/or proliferation of the other microbial strain(s) at a rate that is at least about 5%, 10%, 15%, 20%, 25%, 30%, 35%, 40%, 45%, 50%, 55%, 60%, 65%, 70%, 75%, 80% 85%, 95% 100%, 125%, 150%, 175%, 200%, 250%, 300%, 350%, 400%, 450%, 500%, 600%, 700%, 800%, 900% or more higher than the rate at which the microbial strain from which it was derived produces that same substance. In some embodiments, the enhanced microbial strain releases one or more substances that promote the growth and/or proliferation of the other microbial strain(s) into its environment at a greater rate than does the microbial strain from which it was derived. For example, the enhanced microbial strain may release a substance that promotes the growth and/or proliferation of the other microbial strain(s) at a rate that is at least about 5%, 10%, 15%, 20%, 25%, 30%, 35%, 40%, 45%, 50%, 55%, 60%, 65%, 70%, 75%, 80% 85%, 95% 100%, 125%, 150%, 175%, 200%, 250%, 300%, 350%, 400%, 450%, 500%, 600%, 700%, 800%, 900% or more higher than the rate at which the microbial strain from which it was derived releases that same substance.

Enhanced microbial strains of the present invention may uptake and/or utilize one or more resources (e.g., one or more resources required for the growth and/or proliferation of the enhanced microbial strain and/or or one or more pathogenic microbial strains) more efficiently than the microbial strain from which it was derived. In some embodiments, the enhanced microbial strain uptakes and/or utilizes more carbon, carbon dioxide, glucose, iron, nitrogen (e.g., nitrogen gas), oxygen (e.g., oxygen gas), phosphorous, sulphur, trace metals and/or water than does the microbial strain from which it was derived. For example, the enhanced microbial strain may uptake at least about 5%, 10%, 15%, 20%, 25%, 30%, 35%, 40%, 45%, 50%, 55%, 60%, 65%, 70%, 75%, 80% 85%, 95% 100%, 125%, 150%, 175%, 200%, 250%, 300%, 350%, 400%, 450% or 500% more carbon, carbon dioxide, glucose, iron, nitrogen (e.g., nitrogen gas), oxygen (e.g., oxygen gas), phosphorous, sulphur, trace metals and/or water per cell than the microbial strain from which it was derived. In some embodiments, the enhanced microbial strain uptakes and/or utilizes carbon, carbon dioxide, glucose, iron, nitrogen (e.g., nitrogen gas), oxygen (e.g., oxygen gas), phosphorous, sulphur, trace metals and/or water at a greater rate than does the microbial strain from which it was derived. For example, the enhanced microbial strain may uptake a resource at a rate that is at least about 5%, 10%, 15%, 20%, 25%, 30%, 35%, 40%, 45%, 50%, 55%, 60%, 65%, 70%, 75%, 80% 85%, 95% 100%, 125%, 150%, 175%, 200%, 250%, 300%, 350%, 400%, 450%, 500%, 600%, 700%, 800%, 900% or more higher than the rate at which the microbial strain from which it was derived uptakes that same resource. Similarly, the enhanced microbial strain may utilize a resource at a rate that is at least about 5%, 10%, 15%, 20%, 25%, 30%, 35%, 40%, 45%, 50%, 55%, 60%, 65%, 70%, 75%, 80% 85%, 95% 100%, 125%, 150%, 175%, 200%, 250%, 300%, 350%, 400%, 450%, 500%, 600%, 700%, 800%, 900% or more higher than the rate at which the microbial strain from which it was derived utilizes that same resource.

Enhanced microbial strains of the present invention may uptake and/or utilize one or more resources (e.g., one or more resources required for the growth and/or proliferation of the enhanced microbial strain and one or more pathogenic microbial strains) more efficiently than one or more pathogenic microbial strains. In some embodiments, the enhanced microbial strain uptakes and/or utilizes more carbon, carbon dioxide, glucose, iron, nitrogen (e.g., nitrogen gas), oxygen (e.g., oxygen gas), phosphorous, sulphur, trace metals and/or water than does the pathogenic microbial strain(s). For example, the enhanced microbial strain may uptake at least about 5%, 10%, 15%, 20%, 25%, 30%, 35%, 40%, 45%, 50%, 55%, 60%, 65%, 70%, 75%, 80% 85%, 95% 100%, 125%, 150%, 175%, 200%, 250%, 300%, 350%, 400%, 450% or 500% more carbon, carbon dioxide, glucose, iron, nitrogen (e.g., nitrogen gas), oxygen (e.g., oxygen gas), phosphorous, sulphur, trace metals and/or water per cell than the pathogenic microbial strain(s). In some embodiments, the enhanced microbial strain uptakes and/or utilizes carbon, carbon dioxide, glucose, iron, nitrogen (e.g., nitrogen gas), oxygen (e.g., oxygen gas), phosphorous, sulphur, trace metals and/or water at a greater rate than the pathogenic microbial strain(s). For example, the enhanced microbial strain may uptake a resource at a rate that is at least about 5%, 10%, 15%, 20%, 25%, 30%, 35%, 40%, 45%, 50%, 55%, 60%, 65%, 70%, 75%, 80% 85%, 95% 100%, 125%, 150%, 175%, 200%, 250%, 300%, 350%, 400%, 450%, 500%, 600%, 700%, 800%, 900% or more higher than the rate at which the pathogenic microbial strain(s) uptake(s) that same resource. Similarly, the enhanced microbial strain may utilize a resource at a rate that is at least about 5%, 10%, 15%, 20%, 25%, 30%, 35%, 40%, 45%, 50%, 55%, 60%, 65%, 70%, 75%, 80% 85%, 95% 100%, 125%, 150%, 175%, 200%, 250%, 300%, 350%, 400%, 450%, 500%, 600%, 700%, 800%, 900% or more higher than the rate at which the pathogenic microbial strain(s) utilize(s) that same resource.

Enhanced microbial strains of the present invention may tolerate desiccating conditions better than the microbial strain from which it was derived. In some embodiments, the enhanced microbial strain survives desiccating conditions at a higher rate and/or for longer than the microbial strain from which it was derived. For example, the survival time of the enhanced microbial strain may be at least about 1, 2, 3, 4, 5, 6, 7, 8, 9, 10 or more days longer than the survival rate of the microbial strain from which it was derived. Similarly, the survival rate of the enhanced microbial strain may be at least about 5%, 10%, 15%, 20%, 25%, 30%, 35%, 40%, 45%, 50%, 55%, 60%, 65%, 70%, 75%, 80% 85%, 95% 100%, 125%, 150%, 175%, 200%, 250%, 300%, 350%, 400%, 450%, 500% or more higher than the survival rate of the microbial strain from which it was derived. In some embodiments, the enhanced microbial strain proliferates under desiccating conditions at a higher rate than the microbial strain from which it was derived. For example, the enhanced microbial strain may proliferate at a rate that is at least about 5%, 10%, 15%, 20%, 25%, 30%, 35%, 40%, 45%, 50%, 55%, 60%, 65%, 70%, 75%, 80% 85%, 95% 100%, 125%, 150%, 175%, 200%, 250%, 300%, 350%, 400%, 450%, 500% or more higher than that of the microbial strain from which it was derived. In some embodiments, the enhanced microbial strain proliferates under desiccating conditions to a greater extent than does the microbial strain from which it was derived. For example, the enhanced microbial strain proliferate under desiccating conditions by at least about 5%, 10%, 15%, 20%, 25%, 30%, 35%, 40%, 45%, 50%, 55%, 60%, 65%, 70%, 75%, 80% 85%, 95% 100%, 125%, 150%, 175%, 200%, 250%, 300%, 350%, 400%, 450% or 500% more than the microbial strain from which it was derived.

Enhanced microbial strains of the present invention may tolerate nutrient-deficient conditions (e.g., carbon-deficient conditions, glucose-deficient conditions, nitrogen-deficient conditions, oxygen-deficient conditions and/or phosphorous-deficient conditions) better than the microbial strain from which it was derived, in some embodiments, the enhanced microbial strain survives nutrient-deficient conditions at a higher rate and/or for longer than the microbial strain from which it was derived. For example, the survival time of the enhanced microbial strain may be at least about 1, 2, 3, 4, 5, 6, 7, 8, 9, 10 or more days longer than the survival rate of the microbial strain from which it was derived, Similarly, the survival rate of the enhanced microbial strain may be at least about 5%, 10%, 15%, 20%, 25%, 30%, 35%, 40%, 45%, 50%, 55%, 60%, 65%, 70%, 75%, 80% 85%, 95% 100%, 125%, 150%, 175%, 200%, 250%, 300%, 350%, 400%, 450%, 500% or more higher than the survival rate of the microbial strain from which it was derived. In some embodiments, the enhanced microbial strain proliferates under nutrient-deficient conditions at a higher rate than the microbial strain from which it was derived. For example, the enhanced microbial strain may proliferate at a rate that is at least about 5%, 10%, 15%, 20%, 25%, 30%, 35%, 40%, 45%, 50%, 55%, 60%, 65%, 70%, 75%, 80% 85%, 95% 100%, 125%, 150%, 175%, 200%, 250%, 300%, 350%, 400%, 450%, 500% or more higher than that of the microbial strain from which it was derived. In some embodiments, the enhanced microbial strain proliferates under nutrient-deficient conditions to a greater extent than does the microbial strain from which it was derived. For example, the enhanced microbial strain proliferate under nutrient-deficient conditions by at least about 5%, 10%, 15%, 20%, 25%, 30%, 35%, 40%, 45%, 50%, 55%, 60%, 65%, 70%, 75%, 80% 85%, 95% 100%, 125%, 150%, 175%, 200%, 250%, 300%, 350%, 400%, 450% or 500% more than the microbial strain from which it was derived.

Enhanced microbial strains of the present invention may enhance any suitable aspect of wound healing, including, but not limited to wound hemostasis, wound cell differentiation, time to wound closure and time to wound healing. In some embodiments, the enhanced microbial strain speeds wound hemostasis, increases cellular differentiation, increases fibroblast proliferation, enhances fibroblast activity (e.g., by increasing synthesis of collagen), enhances leukocyte activity (e.g., by increasing antibody production, chemotaxis, cytokine production and/or phagocytosis), speeds wound closure, speeds wound healing, inhibits and/or prevents venous thrombosis, inhibits and/or prevents scar formation, inhibits and/or prevents scar pigmentation, inhibits and/or prevents keloid formation, inhibits and/or prevents the formation of hypertrophic scars (e.g., proud flesh scars in horses), enhances rejection of one or more foreign bodies (e.g., dirt and/or shrapnel) from the wound and/or reduces pain associated with wound healing. In some embodiments, the enhanced microbial strain enhances one or more aspects of wound healing to a greater extent than does the microbial strain from which it was derived. For example, the enhanced microbial strain may enhance an aspect of wound healing by at least about 5%, 10%, 15%, 20%, 25%, 30%, 35%, 40%, 45%, 50%, 55%, 60%, 65%, 70%, 75%, 80% 85%, 95% 100%, 125%, 150%, 175%, 200%, 250%, 300%, 350%, 400%, 450%, 500%, 600%, 700%, 800%, 900% or 1000% more than the microbial strain from which it was derived. In some embodiments, the enhanced microbial strain enhances one or more aspects of wound healing at a greater rate than does the microbial strain from which it was derived. For example, the enhanced microbial strain may enhance an aspect of wound healing at a rate that is at least about 5%, 10%, 15%, 20%, 25%, 30%, 35%, 40%, 45%, 50%, 55%, 60%, 65%, 70%, 75%, 80% 85%, 95% 100%, 125%, 150%, 175%, 200%, 250%, 300%, 350%, 400%, 450%, 500%, 600%, 700%, 800%, 900% or more higher than the rate at which the microbial strain from which it was derived enhances that same aspect of wound healing.

Enhanced microbial strains of the present invention may enhance restoration of the normal microbial environment of a substrate via any suitable means, including, but not limited to, inhibiting and/or preventing colonization by and/or the proliferation of one or more microbial strains (e.g., one or more foreign and/or pathogenic microbial strains), killing one or more microbial strains (e.g., one or more foreign and/or pathogenic microbial strains) and/or promoting colonization by and/or the proliferation of one or more microbial strains (e.g., one or more probiotic microbial strains). In some embodiments, the enhanced microbial strain enhances restoration of the normal microbial environment of a substrate by producing one or more substances that inhibit and/or prevent colonization of a substrate by one or more foreign microbial strains (e.g., one or more foreign and/or pathogenic microbial strains), one or more substances that inhibit and/or prevent proliferation of one or more foreign microbial strains (e.g., one or more foreign and/or pathogenic microbial strains), one or more substances that kill one or more foreign microbial strains (e.g., one or more foreign and/or pathogenic microbial strains), one or more substances that promote colonization of a substrate by one or more microbial strains (e.g., one or more probiotic microbial strains), one or more substances that promote the proliferation of one or more microbial strains (e.g., one or more probiotic microbial strains) and/or one or more substances that inhibit and/or prevent the death of one or more microbial strains (e.g., one or more probiotic microbial strains). In some embodiments, the enhanced microbial strain enhances restoration of the normal microbial environment of a substrate by raising the pH of its environment, the alkalinity of its environment and/or the level of one or more gases (carbon dioxide, methane and/or oxygen) in its environment. In some embodiments, the enhanced microbial strain enhances restoration of the normal microbial environment of a substrate by lowering the pH of its environment, the alkalinity of its environment and/or the level of one or more gases (carbon dioxide, methane and/or oxygen) in its environment. In some embodiments, the enhanced microbial strain enhances restoration of the normal microbial environment of a substrate to a greater extent than does the microbial strain from which it was derived. For example, the enhanced microbial strain may enhance restoration of the normal microbial environment of a substrate by at least about 5%, 10%, 15%, 20%, 25%, 30%, 35%, 40%, 45%, 50%, 55%, 60%, 65%, 70%, 75%, 80% 85%, 95% 100%, 125%, 150%, 175%, 200%, 250%, 300%, 350%, 400%, 450%, 500%, 600%, 700%, 800%, 900% or 1000% more than the microbial strain from which it was derived. In some embodiments, the enhanced microbial strain enhances restoration of the normal microbial environment of a substrate at a greater rate than does the microbial strain from which it was derived. For example the enhanced microbial strain may enhance restoration of the normal microbial environment of a substrate at a rate that is at least about 5%, 10%, 15%, 20%, 25%, 30%, 35%, 40%, 45%, 50%, 55%, 60%, 65%, 70%, 75%, 80% 85%, 95% 100%, 125%, 150%, 175%, 200%, 250%, 300%, 350%, 400%, 450%, 500%, 600%, 700%, 800%, 900% or more higher than the rate at which the microbial strain from which it was derived enhances restoration of the normal microbial environment of that same substrate.

Enhanced microbial strains of the present invention may inhibit and/or prevent sepsis via any suitable means, including, but not limited to, inhibiting and/or preventing colonization of a wound by one or more pathogenic microbial strains, by inhibiting and/or preventing proliferation of one or more pathogenic microbial strains by killing one or more microbial strains (e.g., one or more foreign and/or pathogenic microbial strains), by promoting colonization of a wound by one or more probiotic microbial strains and/or by promoting proliferation of one or more probiotic microbial strains. In some embodiments, the enhanced microbial strain inhibits and/or prevents sepsis by producing one or more substances that inhibit and/or prevent colonization of a substrate by one or more foreign microbial strains (e.g., one or more foreign and/or pathogenic microbial strains), one or more substances that inhibit and/or prevent proliferation of one or more foreign microbial strains (e.g., one or more foreign and/or pathogenic microbial strains), one or more substances that kill one or more foreign microbial strains (e.g., one or more foreign and/or pathogenic microbial strains), one or more substances that promote colonization of a substrate by one or more microbial strains (e.g., one or more probiotic microbial strains), one or more substances that promote the proliferation of one or more microbial strains (e.g., one or more probiotic microbial strains) and/or one or more substances that inhibit and/or prevent the death of one or more microbial strains (e.g., one or more probiotic microbial strains). In some embodiments, the enhanced microbial strain inhibits and/or prevents sepsis by raising the pH of its environment, the alkalinity of its environment and/or the level of one or more gases (carbon dioxide, methane and/or oxygen) in its environment. In some embodiments, the enhanced microbial strain inhibits and/or prevents sepsis by lowering the pH of its environment, the alkalinity of its environment and/or the level of one or more gases (carbon dioxide, methane and/or oxygen) in its environment. In some embodiments, the enhanced microbial strain prevents sepsis to a greater extent than does the microbial strain from which it was derived. For example, the enhanced microbial strain may be at least about 5%, 10%, 15%, 20%, 25%, 30%, 35%, 40%, 45%, 50%, 55%, 60%, 65%, 70%, 75%, 80% 85%, 95% 100%, 125%, 150%, 175%, 200%, 250%, 300%, 350%, 400%, 450%, 500%, 600%, 700%, 800%, 900% or 1000% more effective at preventing sepsis than the microbial strain from which it was derived.

Enhanced microbial strains of the present invention enhance the likelihood survival of a wounded subject via any suitable means, including, but not limited to, enhancing one or more aspects of wound healing and preventing sepsis. In some embodiments, the enhanced microbial strain enhances the likelihood survival of a wounded subject by speeding wound hemostasis, by increasing cellular differentiation, by increasing fibroblast proliferation, by enhancing fibroblast activity (e.g., by increasing synthesis of collagen), by enhancing leukocyte activity (e.g., by increasing antibody production, chemotaxis, cytokine production and/or phagocytosis), by reducing time to wound closure, by reducing time to wound healing, by inhibiting and/or preventing venous thrombosis, by inhibiting and/or preventing scar formation, by inhibiting and/or preventing keloid formation, by inhibiting and/or preventing the formation of hypertrophic scars (e.g., proud flesh scars in horses) and/or by enhancing rejection of one or more foreign bodies (e.g., dirt and/or shrapnel) from the wound. In some embodiments, the enhanced microbial strain enhances the survival rate of wounded subjects to a greater extent than does the microbial strain from which it was derived. For example, the enhanced microbial strain may enhance the survival rate of wounded subjects by at least about 5%, 10%, 15%, 20%, 25%, 30%, 35%, 40%, 45%, 50%, 55%, 60%, 65%, 70%, 75%, 80% 85%, 95% 100%, 125%, 150%, 175%, 200%, 250%, 300%, 350%, 400%, 450%, 500%, 600%, 700%, 800%, 900% or 1000% more than the microbial strain from which it was derived. In some embodiments, the enhanced microbial strain enhances the likelihood of survival of a wounded subject to a greater extent than does the microbial strain from which it was derived. For example, the enhanced microbial strain may enhance the likelihood of survival of a wounded subject by at least about 5%, 10%, 15%, 20%, 25%, 30%, 35%, 40%, 45%, 50%, 55%, 60%, 65%, 70%, 75%, 80% 85%, 95% 100%, 125%, 150%, 175%, 200%, 250%, 300%, 350%, 400%, 450%, 500%, 600%, 700%, 800%, 900% or 1000% more than the microbial strain from which it was derived.

Any suitable method may be used to produce enhanced microbial strains of the present invention, including, but not limited to, the methods described herein.

Enhanced microbial strains of the present invention may be incorporated into any suitable composition, including, but not limited to, animal feed, disinfectants, medicaments and wound dressings.

Medicaments of the present invention may comprise, consist essentially of or consist of one or more enhanced microbial strains (e.g., one or more enhanced microbial strains of the present invention) and a pharmaceutically acceptable excipient.

Medicaments of the present invention may comprise any suitable pharmaceutically acceptable excipient, including, but not limited to, agar, alcohols (e.g., ethyl alcohol), cellulose, cellulosic derivatives (e.g., sodium carboxymethyl cellulose, ethyl cellulose, cellulose acetate, hydroxypropylmethylcellulose and/or hydroxypropylcellulose), esters (e.g., ethyl oleate ethyl laurate and/or sorbitan monostearate), fats (e.g., cocoa butter), fragrances, gelatins, glycols (e.g., propylene glycols), oils (e.g., peanut oil, cottonseed oil, safflower oil, sesame oil, olive oil, corn oil and/or soybean oil), pH adjusting and/or buffering agents, polyols (e.g., glycerine, sorbitol, mannitol and/or polyethylene glycols), preservatives, saline solutions (e.g., isotonic saline and/or phosphate-buffered saline), silicones, starches (e.g., corn starch and/or potato starch), sugar alcohols (e.g., glycerol and/or sorbitol), sugars (e.g., glucose, lactose and/or sucrose), talc, tonicity adjusting and/or buffering agents, tragacanths (e.g., gum tragacanth and/or powdered traganath), water and waxes (e.g., lanolin and/or paraffin). Other examples of pharmaceutically acceptable excipients may be found, for example, in Ansel's PHARMACEUTICAL DOSAGE FORMS AND DRUG DELIVERY SYSTEMS (9th Ed., Lippincott Williams and Wikins (2010)), HANDBOOK OF PHARMACEUTICAL EXCIPIENTS (3rd Ed., American Pharmaceutical Association, Washington, D.C. (2000)), Remington's PHARMACEUTICAL SCIENCES (20th Ed., Mack Publishing, Co., Easton, Pa. (2000)) and REMINGTON: THE SCIENCE AND PRACTICE OF PHARMACY (21st Ed., Lippincott Williams & Wilkins (2005)).

Medicaments of the present invention may comprise any suitable auxiliary substance, including, but not limited to, analgesics agents, anesthetic agents, antibacterial agents, antifungal agents, anti-inflammatory agents, antiviral agents, emollients, growth factors (e.g., epidermal growth factor, keratinocyte growth factor and fibroblast growth factor), hemostatic agents and hormones. Other examples of auxiliary substances may be found, for example. in Ansel's Pharmaceutical Dosage Forms and Drug Delivery Systems (9th Ed., Lippincott Williams and Wikins (2010)), Handbook of Pharmaceutical Excipients (6th Ed., American Pharmaceutical Association (2009)) and Remington: The Science and Practice of Pharmacy (21st Ed., Lippincott Williams & Wilkins (2005)). In some embodiments, the disinfectant comprises one or more substances that disrupt biofilms (e.g., one or more substances that degrades exopolysaccharides).

Medicaments of the present invention may comprise any suitable hemostatic agent, including, but not limited to carbohydrates and iron. In some embodiments, the medicament comprises one or more hemostatic agents selected from the group consisting of collagen, fibrinogen, gelatin, iron, polysaccharides (e.g., cellulose and/or chitosan), platelets, thrombin and/or zeolite. In some embodiments, the medicament comprises one or more hemostatic fine particles (e.g., one or more of the hemostatic particles described, in U.S. Patent Publication No. 2011/0206771). In some embodiments, the medicament comprises one or more hemostatic agents comprising iron (e.g., ferric sulfate). In some embodiments, the medicament comprises one or more styptic agents (e.g., anhydrous aluminium sulfate, potassium alum and/or titanium dioxide).

Medicaments of the present invention may be formulated so as to be suitable for administration via any known method, including, but not limited to, cutaneous, oral, intrarectal, intravaginal, intranasal, intragastric, intratracheal, intravenous and intraventricular. In some embodiments, the medicament is formulated for topical administration (e.g., topical administration at a wound site). In some embodiments, the medicament comprises an aerosol, a cream, an emulsion, a foam, a gel, a lotion, an oil, an ointment, a paste, a powder and/or a spray. Aerosols of liquid particles comprising an enhanced microbial strain (e.g., an enhanced microbial strain of the present invention) can be produced by any suitable means, including, but not limited to, the production of aerosolized liquids using a pressure-driven aerosol nebulizer or an ultrasonic nebulizer. See, e.g., U.S. Pat. No. 4,501,729. Aerosols of solid particles comprising an enhanced microbial strain (e.g., an enhanced microbial strain of the present invention) can likewise be produced by any suitable means, including, but not limited to, the production of aerosolized powders using a solid particulate medicament aerosol generator.

Wound dressings of the present invention may comprise, consist essentially of or consist of one or more enhanced microbial strains (e.g., one or more enhanced microbial strains of the present invention) and a carrier matrix. In some embodiments, the wound dressing comprises a medicament of the present invention. Thus, in some embodiments, the wound dressing comprises a carrier matrix and a medicament comprising one or more enhanced microbial strains of the present invention.

Wound dressings of the present invention may comprise any suitable carrier matrix, including, but not limited to, the carder matrices described in U.S. Pat. Nos. 8,133,484; 8,247,635; 8,258,093; 8,277,837; 8,293,965; 8,299,316; 8,314,283; 8,343,536; 8,343,535; 8,349,354; 8,349,356; 8,350,117; 8,357,402: 8,361,504; 8,362,316; 8,377,015; 8,377,467 and 8,377,468.

Wound dressings of the present invention may comprise any suitable auxiliary substance, including, but not limited to, analgesics agents, anesthetic agents, antibacterial agents, antifungal agents, anti-inflammatory agents, antiviral agents, emollients, growth factors (e.g., epidermal growth factor, keratinocyte growth factor and fibroblast growth factor), hemostatic agents and hormones, pH adjusting and/or buffering agents, preservatives, surfactants and tonicity adjusting and/or buffering agents. In some embodiments, the disinfectant comprises one or more substances that disrupt biofilms (e.g., one or more substances that degrades exopolysaccharides).

Wound dressings of the present invention may comprise any suitable hemostatic agent, including, but not limited to carbohydrates and iron. In some embodiments, the wound dressing comprises one or more hemostatic agents selected from the group consisting of collagen, fibrinogen, gelatin, iron, polysaccharides (e.g., cellulose &idler chitosan), platelets, thrombin and/or zeolite. In sonic embodiments, the wound dressing comprises one or more hemostatic fine particles (e.g., one or more of the hemostatic particles described in U.S. Patent Publication No. 2011/0206771). In some embodiments, the wound dressing comprises one or more hemostatic agents comprising iron (e.g., ferric sulfate). In some embodiments, the wound dressing comprises one or more styptic agents (e.g., anhydrous aluminium sulfate, potassium alum and/or titanium dioxide).

Disinfectants of the present invention may comprise, consist essentially of or consist of one or more enhanced microbial strains (e.g., one or more enhanced microbial strains of the present invention) and a disinfectant carrier.

Disinfectants of the present invention may comprise any suitable disinfectant carrier, including, but not limited to, alcohols, fats, lanolins, oils, polyethylene glycols, saline solutions (e.g., isotonic saline and/or phosphate-buffered saline), silicones, starches, talc, tragacanths (e.g., gum tragacanth), water and waxes (e.g., paraffin).

Disinfectants of the present invention may comprise any suitable formulation, including, but not limited to, aerosols, creams, emulsions, foams, gels, lotions, oils, ointments, pastes, powders and/or sprays. In some embodiments, the disinfectant is formulated for use on human tissue (e.g., skin). In some embodiments, the disinfectant is formulated for use on metal surfaces (e.g., surgical instruments). In some embodiments, the disinfectant is formulated for use on plastic surfaces (e.g., hospital beds). In some embodiments, the disinfectant is formulated for use on porous surfaces (e.g., bed linens). In some embodiments, the disinfectant comprises povidone-iodine.

Disinfectants of the present invention may comprise any suitable auxiliary substance, including, but not limited to, antibacterial agents, antifungal agents, antiviral agents, fragrances, pH adjusting and/or buffering agents, preservatives, surfactants, digestive enzymes (e.g. papain) and tonicity adjusting and/or buffering agents. In some embodiments, the disinfectant comprises one or more substances that disrupt biofilms (e.g., one or more substances that degrades exopolysaccharides).

Animal feed compositions of the present invention may comprise, consist essentially of or consist of one or more enhanced microbial strains (e.g., one or more enhanced microbial strains of the present invention) and a feed matrix.

Animal feed compositions of the present invention may comprise any suitable feed matrix, including, but not limited to, fodder (e.g., grains, hay, legumes, silage and/or straw) and forage (e.g., grass).

Animal feed compositions of the present invention may be fed to any suitable animal, including, but not limited to, farm animals, zoo animals, laboratory animals and/or companion animals. In some embodiments, the animal feed composition is formulated to meet the dietary needs of birds (e.g., chickens, ducks, quails and/or turkeys), bovids (e.g., antelopes, bison, cattle, gazelles, goats, impala, oxen, sheep and/or wildebeests), canines, cervids (e.g., caribou, deer, elk and/or moose), equines (e.g. donkeys, horses and/or zebras), felines, fish, pigs, rabbits, rodents (e.g., guinea pigs, hamsters, mice and/or rats) and the like.

Methods of the present invention may be used to produce any suitable microbial strain, including, but not limited to, enhanced microbial strains of the present invention.

Methods of producing a microbial strain may comprise, consist essentially of or consist of culturing a first microbial strain in the presence of a second microbial strain under conditions sufficient to produce a progeny strain derived from the first microbial strain, wherein the progeny strain possesses at least one desired trait (e.g., at least one desired trait that was absent in the first microbial strain). In some embodiments, the progeny strain is an enhanced microbial strain.

Any suitable culture medium may be used to co-culture the first and second microbial strains, including, but not limited to, complex media, defined media, differential media and selective media. See., e.g., BAUMAN, MICROBIOLOGY WITH DISEASES BY TAXONOMY (Pearson Benjamin Cummings (2007)); GREEN & MOEHLE, MEDIA AND CULTURE OF YEAST, CURRENT PROTOCOLS IN CELL BIOLOGY (Wiley and Sons (2003)); PERRY & STANLEY, MICROBIOLOGY DYNAMICS & DIVERSITY (Saunders College Publishing (1997)). In some embodiments, the first and second microbial strains are co-cultured in a liquid culture medium (e.g., in lysogeny broth or YPD broth). In some embodiments, the first and second microbial, strains are co-cultured on a solid culture medium (e.g., an agar plate). In some embodiments, one or more of the nutrients required for growth and/or proliferation of the first and second microbial strains is present in the medium in a limited amount (e.g., in an amount that is insufficient to support maximal growth/proliferation of both the first and second microbial strains). For example, the culture medium may be nutrient-deficient with respect to carbon, carbon dioxide, glucose, iron, nitrogen (e.g., nitrogen gas), oxygen (e.g., oxygen gas), phosphorous, sulphur, trace metals and/or water. In such embodiments, the natural competition for resources that exists between the first and second microbial strains may be escalated to a non-naturally occurring level, thereby creating selective pressure for an enhanced microbial strain that is able to thrive in the nutrient-deficient medium.

The first and second microbial strains may be co-cultured for any suitable duration. In some embodiments, the first and second microbial strains are co-cultured for a specified period of time (e.g., 6 hours, 12 hours, 24 hours, 36 hours, 48 hours, 72 hours or more). In some embodiments, the first and second microbial strains are co-cultured for a specified number of passages (e.g., 1 passage, 2 passages, 3 passages, 4 passages, 5 passages, 6 passages, 7 passages, 8 passages, 9 passages, 10 passages or more).

The first and second microbial strains may be exposed to any suitable condition, including, but not limited to, agitation, co-culturing with one or additional microbial strains, darkness, a desiccating environment, exposure to ionizing radiation, exposure to one or more digestive enzymes (e.g., papain), exposure to light emitted by a laser (e.g., a low intensity laser), exposure to light emitted by a light emitting diode, exposure to light of a specific wavelength or range of wavelengths, exposure to non-ionizing radiation, exposure to one or inure antibiotics, exposure to one or more oxidation agents, exposure to sound waves of a specific frequency or range of frequencies, nutrient-deficiency, temperature extremes, temperature fluctuations and/or vibration.

The first and second microbial strains may be co-cultured with any suitable microbial strain, including, but not limited to, bacterial strains, mold strains and yeast strains.

The first and second microbial strains may be co-cultured with any suitable bacterial strain, including, but not limited to, probiotic bacterial strains and commensal bacterial strains. In some embodiments, the other microbial strain is derived from a bacterial strain that produces lactic acid. In some embodiments, the other microbial strain is derived from a bacterial strain belonging to a genus selected from the group consisting of Carnobacterium (e.g., C. alterfunditum, C. divergens C. funditum, C. gallinarium, C. inhibens, C. jeotgali, C. maltaromaticum, C. mobile, C. piscicola, C. pleistocenium and/or C. viridans), Enterococcus (e.g., E. aquimarinus, E. asini, E. avium, E. caceae, E. camelliae, E. canintestini, E. canis, E. casseloflavus, E. cecorum, E. columbae, E. devriesei, E. dispar, E. durans, E. faecalis, E, faecium, E. flavescens, E. gallnarum, E. gilvus, F. haemoperoxidus, E. hermanniensis, E. hirae, F. iatlicus, E. lactis, E maodoratus, E. moraviensis, E. mundatii, E. pallens, E. phoeniculicola, E. plantarum, E. porcinus, E. pseudoavium, E. quebecensis, E. raffinosus, E. ratti, E. rivorum, E. saccharolyticus, E. saccharomimimus, E. seriolicida, E. silesiacus, E. solitarius, E. sulfureus, E. termitis, E. thailandicus, E. ureasiticus, E. vikkiensis and/or E. villorum), Lactobacillus (e.g., L. acetotolerans, L. acidifarinae, L. acidipiscis, L. acidophilus, L. agilis, L. algidus, L. alimentarius, L. amylolyticus, L. amylophilus, L. amylotrophicus, L. amylovorus, L. animalis, L. antri, L. apodemi, L. aquaticus, L. arizonensis, L. aviarius, L. bavaricus, L. bifermentans, L. bobalius, L. brantae, L. brevis, L. buchneri, L. bulgarius, L. caaonum, L. camelliae, L. capillatus, L. carnis, L. casei, L. catenaformis, L. cellobiosus, L. ceti, L. coleohominis, L. collinoides, L. composti, L. concavus, L. confusus, L. coryniformis, L. crispatus, L. crustorum, L. curvatus, L. cyprieasei, L. delbrueckii, L. dextrinicus, L. diolovorans, L. divergens, L. durianis, L. equi, L. equicursoris, L. equigenerosi, L. fabifermentans, L. farciminis, L. farraginis, L. ferintoshensis, L. fermentum, L. floricola, L. florum, L. fronicalis, L. fructivorans, L. fructosus, L. frumenti, L. fuchuensis, L. futsaii, L. gallinarium, L. gasseri, L. gastricus, L. ghanensis, L. gigeriorum, L. graminis, L. halotolerans, L. hammesii, L. hamsteri, L. harbinensis, L. hayakitensis, L. helveticus, L. heterochii, L. hilgardii, L. hominis, L. homohiochii, L. hordei, L. iners, L. ingluviei, L. intestinalis, L. jensenii, L. johnsonii, L. kalixensis, L. kandleri, L. kerfiranofaciens, L. kefirgranum, L. kefiri, L. kimchicus, L. kimchii, L. kisonensis, L. kitasatonis, L. koreensis, L. kunkeei, L. lactis, L. leichmannii, L. lindneri, malefermentans, L. mali, L. maltaromicus, L. manihotivorans, L. mindensis, L. minor, L. minutus, L. mucosae, L. murinus, L. nagelii, L. namurensis, L. nantensis, L. nasuensis, L. nodensis, L. odoratitoful, L. oeni, L. oligofermentans, L. oris, L. otakiensis, L. ozensis, L. panis, L. pantheris, L. parabrevis, L. parabuchneri, L. paracasei, L. paracollinoides, L. parafarraginis, L. parakefiri, L. paralimentarius, L. paraplantarum, L. pasteurii, L. paucivorans, L. pentosus, L. perolens, L. pisicola, L. plantarum, L. pobuzihii, L. pontis, L. psittaci, L. rapi, L. rennini, L. reuteri, L. rhamnosus, L. rimae, L. rogosae, L. rossiae, L. ruminis, L. saerimneri, L. sakei, L. salivarius, L. sanfranciscenis, L. saniviri, L. satsumensis, L. secaliphilus, L. selangorensis, L. senioris, L. senmaizukei, L. sharpeae, L. siliginis, L. similis, L. sobrius, L. spicheri, L. sporogenes, L. sucicola, L. suebicus, L. sunkii, L. suntoryeus, L. taiwanensis, L. thailandensis, L. thermotolerans, L. trchodes, L. tucceti, L. uli, L. ultunensis, L. uvarium, L. vaccinostercus, L. vaginalis, L. versmoldensis, L. vini, L. viridescens, L. vitulinus, L. xiangfangensis, L. xylosus, L. yamanashiensis, L. zeae and/or L. zymae) Lactococcus (e.g., L. chugangensis, L. fujiensis, L. garvieae, L. lactis, L. piscium, L. plantarum and/or L. raffinolactis), Leuconostoc (e.g., L. amelibiosum, L. argentinum, L. carnosum, L. citreum, L. cremoris, L. dextranicum, L. durionis, L. fallax, L. ficulneum, L. fructosum, L. gasicomitatum, L. geidum, L. holzapfelii, L. inhae, L. kimchii, L. lactic, L. mesenteroides, L. miyukkimchii, L. oeni, L. palmae, L. paramesenteroides, L. pseudoficulneum and/or L. pseudomesenteroides), Oenococcus (e.g., O. kitaharae and/or O. oeni), Pediococcus (e.g., P. argentinicus, P. cellicola, P. claussenii, P. damnosus, P. dextrinicus, P. ethanolidruans, P. halophilas, P. inopinatus, P. lolli, P. parvulus, P. pentosaceus, P. siamensis, P. stilesii and/or P. urinaeequi), Streptococcus (e.g., S. acidominimus, S. adjacens, S. agalactiae, S. alactolyticus, S. anginosus, S. australius, S. bovis, S. cabalii, S. canis, S. caprinus, S. castoreus, S. cecorum, S. constellatus, S. cremoris, S. criceti, S. cristatus, S. defectivus, S. dentapri, S. dentirousetti, S. devriesei, S. didelphis, S. difficilis, S. downei, S. durans, S. dysgalactiae, S. entericus, S. equi, S. equinus, S. faecalis, S. faecium, S. ferus, S. gallinaceus, S. gallnarium, S. gallolyticus, S. garvieae, S. gordonii, S. halichoeri, S. hansenii, S. henryi, S. hyointestinalis, S. hyovaginalis, S. ictaluri, S. infantarius, S. infantis, S. iniae, S. intermedius, S. intestinalis, S. lactarius, S. lactis, S. lutetiensis, S. macacae, S. macedonicus, S. marimammalium, S. massiliensis, S. merionis, S. minor, S. mitis, S. morbillorum, S. mutans, S. oligofermentans, S. oralis, S. orisratti, S. orisuis, S. ovis, S. parasanguinis, S. parauberis, S. paravuluvs, S. pasteurianus, S. peroris, S. phocae, S. plantarum, S. pleomorphus, S. pluranimalium, S. plurextorum, S. pneumoniae, S. porci, S. porcinus, S. porcorum, S. pseudopneumoniae, S. pseudoporcinus, S. pyogenes, S. raffinolactis, S. ratti, S. rupicaprae, S. saccharolyticus, S. salivarius, S. sanguinis, S. shiloi, S. sinensis, S. sobrinus, S. suis, S. thermophilus, S. thoraltensis, S. tigurinus, S. uberis, S. urinalis, S. ursoris, S. vestibularis and/or S. waius), Tetragenococcus (e.g., T. halophilus, T. koreensis, T. muriaticus, T. osmophilus and/or T. solitarius), Vagoroccus (e.g., V. acidifermentans, V. carniphilus, V. elongatus, V. fessus, V. fluvialis, V. lutrae, V. penaei and/or V. salmoninarum) and Weissel (e.g., W. beninensis, W. ceti, W. cibaria, W. confusa, W. fabaria, W. ghanensis, W. halotolerans, W. hellenica, W. kandleri, W kimchii, W. koreensis, W. minor, W. paramesenteroides, W. soli, W. thailandensis and/or W. viridescens). In some embodiments, the other microbial strain is derived from a bacterial strain belonging to a species selected from the group consisting of Lactobacillus acidophilus, Lactobacillus brevis, Lactobacillus buchneri, Lactobacillus casei, Lactobacillus curvatus, Lactobacillus delbrueckii, Lactobacillus fermentum, Lactobacillus helveticus, Lactobacillus plantarum, Lactobacillus reuteri, Lactobacillus sakei, Lactobacillus sporogenes and Lactobacillus salivarius.

The first and second microbial strains may be co-cultured with any suitable mold strain, including, but not limited to, probiotic mold strains and commensal mold strains.

The first and second microbial strains may be co-cultured with any suitable yeast strain, including, but not limited to, probiotic yeast strains and commensal yeast strains. In some embodiments, the other microbial strain is derived from a yeast strain belonging to a genus selected from the group consisting of Brettanomyces (e.g., B. anomalus, B. bruxellensis, B. custersianus, B. naardenensis and/or B. nanus), Candida (e.g., C. stellata), Dekkera (e.g., D. anomala and/or D. bruxellensis), Saccharomyces (e.g., S. cerevisiae, S. bayanus, S. boulardii and/or S. pastorianus), Schizosaccharomyces (e.g., S. pombe), Torulaspora (e.g., T. delbrueckii), Torulopsis and Zygosaccharomyces (e.g., Z. baili), In some embodiments, the other microbial strain is derived from a yeast strain belonging to a species selected from the group consisting of Brettanomyces anomalus, Brettanomyces bruxellensis, Brettanomyces custersianus, Brettanomyces naardenensis, Brettanomycesnanus, Dekkera anomala, Dekkera bruxellensis, Saccharomyces cerevisiae, Saccharomyces bayanus, Saccharomyces boulardii and Saccharomyces pastorianus.

The first and second microbial strains may be exposed to any suitable antibiotic, including, but not limited to, clindamycin, erythromycin, isoniazid, linezolid, methicillin, penicillin, rifampin, streptomycin, tetracycline and vancomycin.

The first and second microbial strains may be exposed to any suitable type of ionizing radiation, including, but not limited to, alpha particles, beta particles, gamma rays and x-rays.

The first and second microbial strains may be exposed to any suitable type of non-ionizing radiation, including, but not limited to, infrared light, microwaves, radio waves and visible light.

The first and second microbial strains may be exposed to any suitable light, including, but not limited to, infrared light, visible light and ultraviolet light. In some embodiments, the first and second microbial strains are exposed to light having a wavelength in the range of about 10 to about 1000 nm. In some embodiments, the first and second microbial strains are exposed to light having a wavelength in the range of about 10 to about 380 nm. In some embodiments, the first and second microbial strains are exposed to light having a wavelength in the range of about 380 to about 700 nm. In some embodiments, the first and second microbial strains are exposed to light having a wavelength in the range of about 700 to about 1000 nm. In some embodiments, the first and second microbial strains are exposed to light emitted by a laser (e.g., a low intensity “cold” laser). In some embodiments, the first and second microbial strains are exposed to light emitted by a light emitting diode.

The first and second microbial strains may be exposed to any suitable oxidizing agent, including, but not limited to, nitrous oxide, peroxides and ozone.

The first and second microbial strains may be exposed to any suitable sound wave, including, but not limited to, ultrasound waves. In some embodiments, the first and second microbial strains are exposed to sound waves having a frequency of at least about 20 kHz. In some embodiments, the first and second microbial strains are exposed to sound waves having a frequency in the range of about 20 kHz to about 200 MHz.

The first and second microbial strains may be exposed to antibiotics, ionizing radiation, non-ionizing radiation, light, oxidizing agents and/or sound waves for any suitable duration and with any suitable frequency. In some embodiments, the first and second microbial strains are exposed for one or more specified periods of time (e.g., 15 minutes, 30 minutes, 45 minutes, 1 hour, 2 hours, 2 hours, 3 hours, 4 hours, 5 hours, 6 hours, 7 hours, 8 hours, 9 hours, 10 hours, 11 hours, 12 hour, 13 hours, 14 hours, 15 hours, 16 hours, 17 hours, 18 hours, 19 hours, 20 hours, 21 hours, 22 hours, 23 hours, 24 hours, 36 hours, 48 hours, 72 hours or more). In some embodiments, the first and second microbial strains are exposed continuously. In some embodiments, the first and second microbial strains are exposed intermittently (e.g., every half-hour, every hour, every other hour, every 3 hours, every 4 hours, every 5 hours, every 6 hours, every 7 hours, every 8 hours, every 9 hours, every 10 hours, every 11 hours, every 12 hour, every 13 hours, every 14 hours, every 15 hours, every 16 hours, every 17 hours, every 18 hours, every 19 hours, every 20 hours, every 21 hours, every 22 hours, every 23 hours or every 24 hours).

Methods of producing a microbial strain (e.g., an enhanced microbial strain) may further comprise selecting the progeny strain.

Progeny strains (e.g., enhanced microbial strains) may be selected based upon any suitable criteria, including, but not limited to, their ability to inhibit and/or prevent the colonization of a substrate (e.g., human tissue) by one or more pathogenic microbial strains, their ability to inhibit and/or prevent the proliferation of one or more pathogenic microbial strains and/or their ability to kill one or more pathogenic microbial strains. Thus, in some embodiments, the progeny strain is selected based upon its ability to compete with one or more pathogenic microbial strains. In some embodiments, the progeny strain is selected based upon its ability to inhibit and/or prevent the colonization of a wound by one or more pathogenic microbial strains. In some embodiments, the progeny strain is selected based upon its ability to produce one or more inhibitory substances. In some embodiments, the progeny strain is selected based upon its ability to raise the pH of its environment. In some embodiments, the progeny strain is selected based upon its ability to lower the pH of its environment. In some embodiments, the progeny strain is selected based upon its ability to raise the alkalinity of its environment. In some embodiments, the progeny strain is selected based upon its ability to lower the alkalinity of its environment. In some embodiments, the progeny strain is selected based upon its ability to raise the level of one or more gases in its environment (e.g., carbon dioxide, methane and/or oxygen). In some embodiments, the progeny strain is selected based upon its ability to lower the level of one or more gases in its environment (e.g., carbon dioxide, methane and/or oxygen). In some embodiments, the progeny strain is selected based upon its ability to promote the colonization of a substrate (e.g., human tissue) by one or more other microbial strains (e.g., one or more probiotic microbial strains). In some embodiments, the progeny strain is selected based upon its ability to promote the growth and/or proliferation of one or more other microbial strains (e.g., one or more probiotic microbial strains). In some embodiments, the progeny strain is selected based upon its ability to inhibit the death of one or more other microbial strains (e.g., one or more probiotic microbial strains). In some embodiments, the progeny strain is selected based upon its ability to uptake and/or utilize one or more resources (e.g., one or more resources required for the growth and/or proliferation of the pathogenic microbial strain(s)). In some embodiments, the progeny strain is selected based the genotypic differences between it and the microbial strain from which it was derived. In some embodiments, the progeny strain is selected based upon its ability to competitively exclude the microbial strain from which it was derived when the two strains are co-cultured (e.g., when the two strains are co-cultured in a desiccating environment). In some embodiments, the progeny strain is selected based upon its ability to competitively exclude a pathogenic microbial strain when the two strains are co-cultured (e.g., when the two strains are co-cultured in a desiccating environment). In some embodiments, the progeny strain is selected based upon its ability to enhance one or more aspects of wound healing (e.g., wound hemostasis, wound cell differentiation, time to wound closure and/or time to wound healing). For example, in some embodiments, the progeny strain is selected based upon its ability to speed wound hemostasis, to increase cellular differentiation, to increase fibroblast proliferation, to enhance fibroblast activity (e.g., by increasing synthesis of collagen), to enhance leukocyte activity (e.g., by increasing antibody production, chemotaxis, cytokine production and/or phagocytosis), to speed wound closure, to speed wound healing, to inhibit and/or prevent venous thrombosis, to inhibit and/or prevent scar formation, to inhibit and/or prevent scar pigmentation, to inhibit and/or prevent keloid formation, to inhibit and/or prevent the formation of hypertrophic scars (e.g., proud flesh scars in horses), to enhance rejection of one or more foreign bodies (e.g., dirt and/or shrapnel) from the wound and/or to reduce pain associated with wound healing. In some embodiments, the progeny strain is selected based upon its ability to enhance restoration of the normal microbial environment (e.g., to speed restoration of the normal microbial environment following administration of one or more antibioticsantivirals). For example, in some embodiments, the progeny strain is selected based upon its ability to enhance restoration of the normal microbial of the intestinal tract and/or the skin. In some embodiments, the progeny strain is selected based upon its ability to enhance the survival rate of wounded subjects (e.g., gunshot victims). In some embodiments, the progeny strain is selected based upon its ability to enhance the likelihood of survival rate of a wounded subject (e.g., a gunshot victim). In some embodiments, the progeny strain is selected based upon its ability to inhibit and/or prevent sepsis.

Methods of producing a microbial strain (e.g., an enhanced microbial strain) may further comprise culturing the progeny strain with the second microbial strain.

The progeny strain (e.g., the enhanced microbial strain) and the second microbial strain may be co-cultured under any suitable conditions, including, but not limited to, the conditions described above with respect to the co-culturing of the first and second microbial strains. Thus, in some embodiments, the progeny strain is co-cultured with the second microbial strain under conditions that are the same (or substantially the same) as the conditions under which it was originally derived.

Methods of producing a microbial strain (e.g., an enhanced microbial strain) may be iterative processes giving rise to progeny strains (e.g., enhanced microbial strains) having numerous desired traits. For example, a first microbial strain may be co-cultured with a second microbial strain to produce a first progeny strain, which may be co-cultured with a third microbial strain to produce a second progeny strain, which may be co-cultured with a fourth microbial strain to produce a third progeny strain. Thus, methods of the present invention may be used to produce progeny strains (e.g., enhanced microbial strains) capable of inhibiting and/or preventing the growth and/or proliferation of numerous pathogenic microbial strains.

Methods of producing a microbial strain (e.g., an enhanced microbial strain) may further comprise maintaining the progeny strain (e.g., the enhanced microbial strain) under conditions sufficient to promote the retention of one or more desired straits (e.g., the ability to inhibit and/or prevent the proliferation of one or more pathogenic microbial strains). In some embodiments, the progeny strain is maintained under ideal growth conditions to minimize and/or eliminate selective pressures, thereby reducing the likelihood that the progeny strain will mutate in such a way that its desired trait(s) is/are lost.

Methods of the present invention may be used to inhibit and/or prevent the colonization of a substrate by one or more pathogenic microbial strains, to inhibit and/or prevent proliferation of one or more pathogenic microbial strains on a substrate and/or to kill one or more pathogenic microbial strains on a substrate. Such methods may comprise, consist essentially of or consist of applying a microbial strain (e.g., an enhanced microbial strain) to the substrate in an amount sufficient to inhibit and/or prevent the colonization of the substrate by one or more pathogenic microbial strains, to inhibit and/or prevent proliferation of one or more pathogenic microbial strains on the substrate and/or to kill one or more pathogenic microbial strains on the substrate.

Methods of the present invention may he used to treat any suitable disease or disorder, including, but not limited to, diseases/disorders caused by one or more pathogenic microbial strains. In some embodiments, methods of the present invention are used to treat diseases/disorders caused by a bacterial strain that is at least partially resistant to one or more antibiotics. For example, in some embodiments, methods of the present invention arc used to treat diseases/disorders caused by one or more bacterial strains selected from the group consisting of clindamycin-resistant Clostridium difficile, fluoroquinolone-resistant Clostridium difficile, fluoroquinolone-resistant Escherichia coli, fluoroquinolone-resistant Salmonella, isoniazid-resistant Mycobacterium tuberculosis, linezolid-resistant Enterococcus facealis, linezolid-resistant Enterococcus faecium, macrolide-resistant Streptococcus pyogenes, methicillin-resistant Staphylococcus aureus, methicilin-resistant Staphylococcus epidermidis, multidrug-resistant Acinetobacter baumannii, multidrug-resistant Enterococcus faecalis, multidrug-resistant Enterococcus faecium, penicillin-resistant Streptococcus pneumoniae, penicillin-resistant Enterococcus faecalis, penicillin-resistant Enterococcus faecium, rifampin-resistant Mycobacterium tuberculosis, streptomycin-resistant Mycobacterium tuberculosis, vancomycin-resistant Enterococcus faecalis, vancomycin-resistant Enterococcus faecium, vancomycin-resistant Escherichia coli and vancomycin-resistant Staphylococcus aureus.

Methods of treating a disorder in a subject in need thereof may comprise, consist essentially of or consist of administering to said subject a therapeutically effective amount of a microbial strain (e.g., an enhanced microbial strain of the present invention).

Methods of the present invention may be used to enhance one or more aspects of wound healing. Such methods may comprise, consist essentially of or consist of applying a microbial strain (e.g., an enhanced microbial strain) to a wound in an amount sufficient to enhance one or more aspects of wound healing.

Methods of the present invention may be used to enhance any suitable aspect of wound healing, including, but not limited to, wound hemostasis, wound cell differentiation, time to wound closure and/or time to wound healing. For example, in some embodiments, the methods are used to speed wound hemostasis, to increase cellular differentiation, to increase fibroblast proliferation, to enhance fibroblast activity (e.g., by increasing synthesis of collagen), to enhance leukocyte activity (e.g., by increasing antibody production, chemotaxis, cytokine production and/or phagocytosis), to speed wound closure, to speed wound healing, to inhibit and/or prevent venous thrombosis, to inhibit and/or prevent scar formation, to inhibit and/or prevent scar pigmentation, to inhibit and/or prevent keloid formation, to inhibit and/or prevent the formation of hypertrophic scars (e.g., proud flesh scars in horses), to enhance rejection of one or more foreign bodies (e.g., dirt and/or shrapnel) from the wound and/or to reduce pain associated with wound healing. In some embodiments, the methods may be used to enhance one or more aspects of wound healing by at least about 5%, 10%, 15%, 20%, 25%, 30%, 35%, 40%, 45%, 50%, 55%, 60%, 65%, 70%, 75%, 80% 85%, 95% 100%, 125%, 150%, 175%, 200%, 250%, 300%, 350%, 400%, 450% or 500% or more e.g., as compared to wound healing in one or more control subjects and/or as compared to wound healing of an untreated wound in said subject).

Methods of enhancing one or more aspects of wound healing in a subject in need thereof may comprise, consist essentially of or consist of administering to said subject a therapeutically effective amount of a microbial strain (e.g., an enhanced microbial strain of the present invention).

Methods of the present invention may be used to enhance restoration of the normal microbial environment of a substrate. Such methods may comprise, consist essentially of or consist of applying a microbial strain (e.g., an enhanced microbial strain) to the substrate in an amount sufficient to enhance restoration of the normal microbial environment.

Methods of the present invention may be used to restore the normal microbial environment of any suitable substrate, including, but not limited to, human tissue. For example, in some embodiments, the methods are used to enhance restoration of the normal microbial environment of a wound following administration of one or more antibioticsantivirals to a human subject.

Methods of the present invention may be used to restore the normal microbial environment by any suitable means, including, but not limited to, by inhibiting and/or preventing colonization by and/or the proliferation of one or more microbial strains (e.g., one or more foreign and/or pathogenic microbial strains), by killing one or more microbial strains (e.g., one or more foreign and/or pathogenic microbial strains) and/or by promoting colonization by and/or the proliferation of one or more microbial strains (e.g., one or more probiotic microbial strains). For example, in some embodiments, the methods are used to enhance restoration of the normal microbial environment by producing one or more substances that inhibit and/or prevent colonization by and/or the proliferation of one or more foreign microbial strains (e.g., one or more foreign and/or pathogenic microbial strains), by producing one or more substances that kill one or more foreign microbial strains (e.g., one or more foreign and/or pathogenic microbial strains), by producing one or more substances that promote colonization by and/or the proliferation of one or more microbial strains (e.g., one or more probiotic microbial strains), by producing one or more substances that inhibit and/or prevent the death of one or more microbial strains (e.g., one or more probiotic microbial strains), by raising the pH of the environment, by lowering the pH of the environment, by raising the alkalinity of the environment, by lowering the alkalinity of the environment, by raising the level of one or more gases (carbon dioxide, methane and/or oxygen) in the environment and/or by lowering the level of one or more gases (carbon dioxide, methane and/or oxygen) in the environment. In some embodiments, the methods may be used to enhance restoration of the normal microbial environment by at least about 5%, 10%, 15%, 20%, 25%, 30%, 35%, 40%, 45%, 50%, 55%, 60%, 65%, 70%, 75%, 80% 85%, 95% 100%, 125%, 150%, 175%, 200%, 250%, 300%, 350%, 400%, 450% or 500% or more (e.g., as compared to restoration of the normal microbial environment of one or more control substrates).

Methods of enhancing restoration of the normal microbial environment in a subject in need thereof may comprise, consist essentially of or consist of administering to said subject a therapeutically effective amount of a microbial strain (e.g., an enhanced microbial strain of the present invention).

Methods of the present invention may be used to inhibit and/or prevent sepsis. Such methods may comprise, consist essentially of or consist of a microbial strain (e.g., an enhanced microbial strain) to a wound in an amount sufficient to inhibit and/or prevent sepsis.

Methods of the present invention may be used to inhibit and/or prevent sepsis via any suitable means, including, but not limited to, by inhibiting and/or preventing colonization of a wound by one or more pathogenic microbial strains, by inhibiting and/or preventing proliferation of one or more pathogenic microbial strains by killing one or more microbial strains (e.g., one or more foreign and/or pathogenic microbial strains), by promoting colonization of a wound by one or more probiotic microbial strains and/or by promoting proliferation of one or more probiotic microbial strains. For example, in some embodiments, the methods are used to inhibit and/or prevent sepsis by producing one or more substances that inhibit and/or prevent colonization by and/or the proliferation of one or more foreign microbial strains (e.g., one or more foreign and/or pathogenic microbial strains), by producing one or more substances that kill one or more foreign microbial strains (e.g., one or more foreign and/or pathogenic microbial strains), by producing one or more substances that promote colonization by and/or the proliferation of one or more microbial strains (e.g., one or more probiotic microbial strains), by producing one or more substances that inhibit and/or prevent the death of one or more microbial strains (e.g., one or more probiotic microbial strains), by raising the pH of the environment, by lowering the pH of the environment, by raising the level of one or more gases (carbon dioxide, methane and/or oxygen) in the environment and/or by lowering the level of one or more gases (carbon dioxide, methane and/or oxygen) in the environment. In some embodiments, the methods may be used to inhibit the onset of sepsis by at least about 5%, 10%, 15%, 20%, 25%, 30%, 35%, 40%, 45%, 50%, 55%, 60%, 65%, 70%, 75%, 80% 85%, 95%, 96%, 97%, 98%, 99% or 100% or more (e.g., as compared to sepsis onset in one or more control subjects). In some embodiments, the methods may be used to inhibit the progression of sepsis by at least about 5%, 10%, 15%, 20%, 25%, 30%, 35%, 40%, 45%, 50%, 55%, 60%, 65%, 70%, 75%, 80% 85%, 95%, 96%, 97%, 98%, 99% or 100% or more (e.g., as compared to sepsis progression in one or more control subjects).

Methods of inhibiting and/or preventing sepsis in a subject in need thereof may comprise, consist essentially of or consist of administering to said subject a therapeutically effective amount of a microbial strain (e.g., an enhanced microbial strain of the present invention).

Methods of the present invention may be used to enhance the likelihood of survival of a subject (e.g., a wounded subject). Such methods may comprise, consist essentially of or consist of applying a microbial strain (e.g., an enhanced microbial strain) to a wound in an amount sufficient to enhance the likelihood of survival of the subject.

Methods of the present invention may be used to enhance the likelihood of survival of a subject having any suitable type of wound, including, but not limited to, a gunshot wound, a knife wound, a shrapnel wound and a surgical wound.

Methods of the present invention may be used to enhance the likelihood of survival of subject (e.g., a wounded subject) via any suitable means, including, but not limited to, enhancing one or more aspects of wound healing and preventing sepsis. For example, in some embodiments, the methods are used to speed wound hemostasis, to increase cellular differentiation, to increase fibroblast proliferation, to enhance fibroblast activity (e.g., by increasing synthesis of collagen), to enhance leukocyte activity (e.g., by increasing antibody production, chemotaxis, cytokine production and/or phagocytosis), to speed wound closure, to speed wound healing, to inhibit and/or prevent venous thrombosis, to inhibit and/or prevent scar formation, to inhibit and/or prevent scar pigmentation, to inhibit and/or prevent keloid formation, to inhibit and/or prevent the formation of hypertrophic scars (e.g., proud flesh scars in horses), to enhance rejection of one or more foreign bodies (e.g., dirt and/or shrapnel) from the wound and/or to reduce pain associated with wound healing. In some embodiments, the methods may be used to enhance the likelihood of survival of a subject by at least about 5%, 10%, 15%, 20%, 25%, 30%, 35%, 40%, 45%, 50%, 55%, 60%, 65%, 70%, 75%, 80% 85%, 95% 100%, 125%, 150%, 175%, 200%, 250%, 300%, 350%, 400%, 450% or 500% or more (e.g., as compared to the survival rate of untreated subjects).

Methods of enhancing the likelihood of survival of a subject (e.g., a wounded subject) may comprise, consist essentially of or consist of administering to said subject a therapeutically effective amount of a microbial strain (e.g., an enhanced microbial strain of the present invention). In sonic embodiments, administering the microbial strain comprises applying the microbial strain to a wound.

Microbial strains (e.g., enhanced microbial strains of the present invention) may be administered using any suitable method known in the art, including, but not limited to, cutaneous, oral, intrarectal, intravaginal, intranasal, intragastric and intratracheal delivery. In some embodiments, the microbial strain is administered directly into a wound site.

Microbial strains (e.g., enhanced microbial strains of the present invention) may be administered in any suitable dosage.

Those skilled in the art will appreciate how to select the appropriate dosage(s) and route(s) of administration based upon such factors as the disorder being treated, the subject's age, the subject's general health, the subject's history of previous infections, the type and/or size of the wound(s) being treated, etc.

### EXAMPLES

The following examples are not intended to be a detailed catalogue of all the different ways in which the present invention may be implemented or of all the features that may be added to the present invention. Subjects skilled in the art will appreciate that numerous variations and additions to the various embodiments may be made without departing from the present invention. Hence, the following descriptions are intended to illustrate some particular embodiments of the invention, and not to exhaustively specify all permutations, combinations and variations thereof.

### Example 1

### Enhanced Yeast Strain for the Treatment and Prevention of Methicillin-Resistant Staph Infections

Saccharomyces boulardii and methicillin-resistant Staphylococcus aureus (MRSA) are co-cultured on nutrient-deficient agar plates (i.e., agar plates comprising an amount of one or more nutrients (e.g., carbon, glucose, nitrogen, phosphorous and/or water) that is limiting insofar as it is insufficient to support maximal growth of S. boulardii and MRSA simultaneously) for 3 days at 37° C. Individual colonies of S. boulardii are passaged to fresh nutrient-deficient agar plates, which are subsequently inoculated with MRSA and then incubated for 3 days at 37° C. The plates are divided into two groups based upon the amount of MRSA proliferation: high-growth MRSA plates and low-growth MRSA plates. Control agar plates (i.e., agar plates comprising a non-limiting amount of nutrients) are inoculated with individual colonies of S. boulardii taken from high-growth MRSA plates individual colonies of S. boulardii taken from low-growth MRSA plates and incubated for 3 days at 37° C. Following the incubation period, the now S. boulardii-laden control plates are inoculated with fresh MRSA and incubated for an additional 3 days at 37° C. Control plates comprising S. boulardii colonies taken from high-growth MRSA plates are flush with MRSA, whereas control plates taken from low-growth MRSA plates are devoid of MRSA and/or have markedly reduced MRSA proliferation.

### Example 2

### Enhanced Bacterial Strain for the Treatment and Prevention f Methicillin-Resistant Staph Infections

Lactobacillus brevis and methicillin-resistant Staphylococcus epidermis are co-cultured on agar plates comprising lysogeny broth (hereinafter “LB plates”) for 3 days at 37° C. under desiccating conditions. Individual colonies of L. brevis are passaged to fresh LB plates, which are subsequently inoculated with methicillin-resistant. S. epidermis (MRSE) and then incubated for 3 days at 37° C. under desiccating conditions. The plates are divided into two groups based upon the amount of MRSE proliferation: high-growth MRSE plates and low-growth MRSE plates. Fresh LB plates are inoculated with individual colonies of L. brevis taken from high-growth MRSE plates individual colonies of L. brevis taken from low-growth MRSE plates and incubated for 3 days at 37° C. Following the incubation period, the now S. boulardii-laden control plates are inoculated with fresh MRSE and incubated for an additional 3 days at 37° C. Control plates comprising L. brevis colonies taken from high-growth MRSE plates are flush with MRSE, whereas control plates taken from low-growth MRSE plates are devoid of MRSE and/or have markedly reduced MRSE proliferation.

The foregoing is illustrative of the present invention, and is not to be construed as limiting thereof The invention is defined by the following claims, with equivalents of the claims to be included herein.

1. An enhanced microbial strain having to the ability to inhibit and/or prevent colonization of a substrate by a second microbial strain, to inhibit and/or prevent proliferation of the second microbial strain, to kill the second microbial strain, to produce one or more inhibitory substances, to raise the pH of its environment, to lower the pH of its environment, to raise the alkalinity of its environment, to lower the alkalinity of its environment, to raise the level of one or more gases in its environment, to lower the level of one or more gases in its environment, to promote the colonization of the substrate by a third microbial strain, to promote the proliferation of the third microbial strain, to inhibit and/or prevent the death of the third microbial strain, to uptake and/or utilize one or more resources required for proliferation of the second microbial strain at a greater rate than the second microbial strain, to survive and/or proliferate in a desiccating environment, to survive and/or proliferate in a nutrient-deficient environment, to enhance one or more aspects of wound healing, to enhance restoration of a normal microbial environment, to prevent sepsis, and/or to enhance the likelihood of survival of a wounded subject.

2-3. (canceled)

4. The method of claim 1, wherein said substrate comprises human tissue.

5. The method of claim 1, wherein said one or more inhibitory substances comprises one more substances selected from the group consisting of: alcohols, antibiotics, beta lactams, biofilm disruption molecules, carbon dioxide, proliferation inhibitors and/or a toxic polypeptides.

6. The method of claim 1, wherein said third microbial strain is a probiotic microbial strain, the proliferation of which inhibits and/or prevents the colonization of the substrate b the second microbial strain and/or the proliferation of the second microbial strain.

7-8. (canceled)

9. The method of claim 1, wherein said one or more resources required for proliferation of the second microbial strain comprises one or more resources selected from the group consisting of carbon, carbon dioxide, glucose, iron, nitrogen, oxygen, phosphorous, sulphur, trace metals and water.

10-15. (canceled)

16. The method of claim 1, wherein the enhanced microbial strain is a yeast strain.

17. (canceled)

18. The method of claim 1, wherein the enhanced microbial strain is a yeast strain belonging to a species selected from the group consisting of Saccharomyces cerevisiae, Saccharomyces bayanus, Saccharomyces boulardii and Saccharomyces pastorianus.

19. The method of claim 1, wherein the enhanced microbial strain is a bacterial strain.

20. (canceled)

21. The method of claim 1, wherein the enhanced microbial strain is a bacterial strain belonging to a species selected from the group consisting of Lactobacillus acidophilus, Lactobacillus brevis, Lactobacillus buchneri, Lactobacillus casei, Lactobacillus curvatus, Lactobacillus delbrueckii, Lactobacillus fermentum, Lactobacillus helveticus, Lactobacillus plantarum, Lactobacillus reuteri, Lactobacillus sakei, Lactobacillus sporogenes and Lactobacillus salivarius.

22-25. (canceled)

26. The method of claim 1, wherein the second microbial strain is a pathogenic bacterial strain that is at least partially resistant to one or more antibiotics.

27. (canceled)

28. The method of claim 1, wherein the second microbial strain is a methicillin-resistant strain of Staphylococcus aureus or Staphylococcus epidermidis.

29-65. (canceled)

66. A medicament comprising an enhanced microbial strain according to claim 1.

67-72. (canceled)

73. A wound dressing comprising an enhanced microbial strain according to claim 1.

74-75. (canceled)

76. A disinfectant comprising an enhanced microbial strain according to claim 1.

77-78. (canceled)

79. A method comprising:
applying an enhanced microbial strain according to claim 1 to a substrate in an amount effective to inhibit and/or prevent colonization of the substrate by one or more pathogenic microbial strains, to inhibit and/or prevent proliferation of one or more pathogenic microbial strains on the substrate, and/or to kill one or more pathogenic microbial strains on the substrate.

80-104. (canceled)

105. A method of treating a disorder caused by one or more pathogenic microbial strains in a subject in need thereof, comprising:
administering to said subject a therapeutically effective amount of an enhanced microbial strain according to claim 1.

106-110. (canceled)

111. The method of claim 105, wherein said one or more pathogenic microbial strains comprises a methicillin-resistant strain of Staphylococcus aureus or Staphylococcus epidermidis.

112-120. (canceled)

121. A method of enhancing the likelihood of survival of a wounded subject, comprising:
administering to said subject a therapeutically effective amount of an enhanced microbial strain according to claim 1.

122. A method of inhibiting and/or preventing sepsis in a subject in need thereof, comprising:
administering to said subject a prevention effective amount of an enhanced microbial strain according to claim 1.

123-128. (canceled)

129. A method of enhancing one or more aspects of wound healing in a subject in need thereof, comprising:
applying a therapeutically effective amount of an enhanced microbial strain according to claim 1 to a wound.

130-142. (canceled)
